(19) United States
(12) Patent Application Publication
(10) Pub. No.: US 2026/0152804 A1
(43) Pub. Date: Jun. 4, 2026

(54) MATERIALS AND METHODS FOR DETERMINING GAIN OR LOSS OF MRNA TRANSCRIPTS

(71) Applicant: VENTANA MEDICAL SYSTEMS, INC., Tucson, AZ (US)

(72) Inventors: Martin Anastasios Asimis, Cambridge, MA (US); Jean Z. Boyer, Tucson, AZ (US); Azita Djalilvand, Tucson, AZ (US); Erica Lynn Harnish, Byron, MN (US); Robert Kraft, Tucson, AZ (US); Lizhen Pang, Tucson, AZ (US); Shaowu Tang, Pleasanton, CA (US)

(21) Appl. No.: 19/415,905

(22) Filed: Dec. 11, 2025

Related U.S. Application Data
(63) Continuation of application No. PCT/US2024/037249, filed on Jul. 9, 2024.
(60) Provisional application No. 63/513,310, filed on Jul. 12, 2023.

(51) Int. Cl. C12Q 1/6886; G16B 25/10; G16B 40/20

(52) U.S. Cl. CPC C12Q 1/6886; G16B 25/10; G16B 40/20; C12Q 2600/158

(57) ABSTRACT

Methods of evaluating mRNA or cDNA are provided. Instead of evaluating the mRNA or cDNA as a whole, multiple distinct portions of the mRNA or cDNA (such as different exons or untranslated regions) are separately quantified. The separate quantities are then evaluated by a trained classifier, which converts the separate quantities to a composite score indicative of the expression level of the mRNA or corresponding cDNA.

### CROSS REFERENCE TO RELATED APPLICATIONS

The present application is a continuation of International Application No. PCT/US2024/037249, filed on Jul. 9, 2024, which application claims the benefit of the filing date of U.S. Provisional Patent Application No. 63/513,310 filed on Jul. 12, 2023, the disclosures of which are hereby incorporated by reference herein in their entireties.

### FIELD OF THE DISCLOSURE

Detection and quantification of mRNA transcripts and/or corresponding cDNA, especially as applied for determining an expression status of an encoded protein and/or diagnostic status of a subject.

### SEQUENCE LISTING INCORPORATION BY REFERENCE

This application hereby incorporates by reference a sequence listing submitted herewith in XML format, having a file name of P38485US_SeqList, created on Jan. 13, 2024, which is 9,175 bytes in size.

### BACKGROUND OF THE DISCLOSURE

Many different methodologies exist for identifying and quantifying mRNA in biological samples.

Geiss et al. describe a method for direct detection of mRNA in solution using uniquely coded fluorescently labeled probes. A “reporter probe” comprises a linearized single-stranded DNA “backbone” segment. Four 15-base repeats are ligated to the 5′ end of the backbone and a 35-50 base target-specific sequence is ligated to the 3′ end of the backbone. Fluorescently labeled, in vitro transcribed RNA segments are annealed to the backbone to form a double-stranded section between the single stranded target specific segment at 3′ end and the single stranded repeat segment at the 5′ end. The RNA segments of each target specific probe have a unique pattern of fluorescent labels that provides a unique “signal” associated with the target-specific segment. A “capture probe” is also provided that comprises (a) at the 5′ end a single-stranded 35- to 50-base mRNA-specific DNA sequence that hybridizes to a non-overlapping region of the same target as the reporter probe and; (b) at 3′ end, two 15-base repeats linked to a biotin molecule. In use, the reporter and capture probes are hybridized to the target nucleic acid molecule to form a tripartite capture-target-reporter complex. The tripartite complex is then affinity purified via 3′ repeat region and the 5′ repeat region to remove excess reporter and capture probes. The purified complex is then attached to a streptavidin-coated surface via the 3′ repeat. A voltage is applied to elongate and align the molecules and a biotinylated oligonucleotide is hybridized to the 5′ repeat, thereby immobilizing the reporter probe to the streptavidin-coated surface. The immobilized reporters are then imaged and counted. Materials and methods useful for practicing this concept and variations of the process are disclosed in, for example, WO 2007-076128 A2 and WO 2010-019826 A1.

WO 2017-015099 A1 discloses a method of identifying and quantifying mRNA in tissue samples using uniquely coded fluorescently labeled probes. A nucleic acid probe is provided comprising a target-binding domain and a signal oligonucleotide separated from one another by a cleavable linker. The signal oligonucleotide has a unique pattern of fluorescent labels that provides a unique “signal” associated with the target-binding domain. The nucleic acid probe is contacted with a tissue section under conditions that permit specific hybridization between the target-binding domain and its corresponding target in the tissue sample. Specific regions of interest (ROIs) of the tissue section are then subjected to cleavage to release the signal oligonucleotides from the bound probes and the signal oligonucleotides are collected and quantitated. The process can be repeated at multiple ROIs throughout the tissue sample to generate transcript counts specific for each ROI. Moreover, a composite image with the RNA expression data may be generated by overlaying the expression data for each ROI on a digital image of the tissue section. See also Zollinger et al.

WO 2012-178046 A2 discloses a method of performing multivariate gene expression analysis. A probe set is provided comprising a plurality of probes against a set of mRNA of interest and a set of housekeeping mRNA. A test RNA sample is provided, derived from a sample in which the status of the genes is to be determined. A reference RNA sample is also provided, which comprises equimolar quantities of each of the mRNA of interest. Hybridization between the probe set and the target mRNAs is detected and quantitated in the test sample and in the reference sample according to the method of Geiss. The gene counts for the mRNA of interest in the test sample are normalized the geometric mean of the housekeeping genes in the test sample to obtain normalized test sample gene counts. The gene counts for the mRNA of interest in the reference sample are divided by the geometric mean of the housekeeping genes in the test sample to obtain normalized reference sample gene counts. The normalized test sample gene counts are then divided by the normalized reference sample gene counts to obtain final normalized gene counts. The final normalized gene counts are then entered into an algorithm to generate a test result.

In each of the foregoing methods, a single “count” is generated for the specific gene transcript. However, this may give variable results relative to each other, demonstrating that any single probe (or probe pair) is not the most reliable measure of the expression level of a gene.

### SUMMARY OF THE DISCLOSURE

The present disclosure relates to methods of evaluating test samples, where multiple regions of the same mRNA transcript are quantitated and then evaluated by a trained classifier to assign the sample a protein status or diagnostic status.

In an embodiment, a method of determining an expression status of a protein in a test sample and/or a diagnostic status of the test sample is provided, the method comprising: obtaining a normalized quantity for each of at least 2 distinct regions of a messenger ribonucleic acid (mRNA) transcript encoding the protein of interest in a test sample (NTest); obtaining either: a normalized reference quantity for each of the at least 2 distinct regions of the mRNA transcript in a reference sample (NRef); or a non-normalized quantity of each of the at least 2 distinct regions of the mRNA in a reference sample (QRef), wherein the reference sample comprises a known quantity of the mRNA; for each of the distinct regions of the mRNA, generating a ratio between the NTest and either NRef or QRef to obtain a set of region ratios; and applying the set of region ratios to a trained classifier to obtain a score indicative of the expression status of the protein and/or the diagnostic status.

In another exemplary embodiment, a method is provided comprising: (a) performing a quantitative or semi-quantitative nucleic acid detection method on a test sample comprising mRNA or cDNA, wherein the quantitative or semi-quantitative nucleic acid detection method comprises: (a1) contacting the test sample with a probe set comprising: (a1a) a plurality of distinctly labeled probes or probe pairs specific for distinct regions of the same mRNA or corresponding cDNA (distinct mRNA regions); and (a1b) one or more distinctly labeled probes or probe pairs specific for a housekeeping mRNA or corresponding cDNA; and (a2) detecting and quantifying each probe or probe pair that specifically hybridizes to the distinct mRNA regions to obtain a quantity (Q_{Test}); and (a3) detecting and quantifying each probe or probe pair that specifically hybridizes to the housekeeping mRNA to obtain a quantity (HK_{Test}); and (b) performing a quantitative or semi-quantitative nucleic acid detection method on a reference sample comprising mRNA or cDNA, wherein the quantitative or semi-quantitative nucleic acid detection method comprises: (b1) contacting the reference sample with the probe set; (b2) detecting and quantifying each probe or probe pair that specifically hybridizes to the distinct mRNA regions to obtain a quantity (Q_{Ref}); and (b3) detecting and quantifying each probe or probe pair that specifically hybridizes to the housekeeping mRNA to obtain a quantity (HK_{Ref}).

In another exemplary embodiment, a method is provided comprising: (a) performing a quantitative or semi-quantitative nucleic acid detection method on a test sample comprising mRNA or cDNA, wherein the quantitative or semi-quantitative nucleic acid detection method comprises: (a1) contacting the test sample with a probe set comprising: (a1a) a plurality of distinctly labeled probes or probe pairs specific for distinct regions of an mRNA of interest or a corresponding cDNA (distinct mRNA regions); and (a1b) one or more distinctly labeled probes or probe pairs specific for a housekeeping mRNA or corresponding cDNA; and (a2) detecting and quantifying each probe or probe pair that specifically hybridizes to the distinct mRNA regions to obtain a quantity (Q_{Test}); and (a3) detecting and quantifying each probe or probe pair that specifically hybridizes to the housekeeping mRNA to obtain a quantity (HK_{Test}); and (b) performing a quantitative or semi-quantitative nucleic acid detection method on a reference sample comprising a known quantity of the mRNA of interest or the corresponding cDNA, wherein the quantitative or semi-quantitative nucleic acid detection method comprises: (b1) contacting the reference sample with the plurality of distinctly labeled probes or probe pairs specific for distinct mRNA regions; and (b2) detecting and quantifying each probe or probe pair that specifically hybridizes to the distinct mRNA regions to obtain a quantity (Q_{Ref}).

Additional embodiments and features of the disclosure are described in more detail below.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A is a workflow of one exemplary embodiment of the methods described herein.

FIG. 1B is a workflow of one exemplary embodiment of the methods described herein.

FIG. 2 illustrates locations within a canonical phosphatase and tensin homologue (PTEN) transcript against which probe sets were designed. Each of the dark bars at the bottom illustrates a 100 nucleotide sequence against which capture probes and reporter probes are designed for use in a NANOSTRING NCOUNTER assay.

FIG. 3A illustrates the relative expression levels detected by the PTEN probes in RNA extracted from tumor-derived cell lines to demonstrate the functionality and specificity of the probe set. Normalized counts are shown representing transcript abundance for each PTEN probe for each cell line. Cell line NCI-H460 has high PTEN protein abundance and Calu-3 has low PTEN protein abundance which is consistent with the relative levels of RNA detected for these 2 cell lines by all 9 probes. Although PTEN protein is absent in LNCaP, RNA expression by all 9 probes is still detected. Normalized counts shown for all probes are the mean of duplicate samples.

FIG. 3B illustrates the relative expression levels detected by the PTEN probes in RNA extracted from tumor-derived cell lines to demonstrate the functionality and specificity of the probe set. Normalized counts are shown representing transcript abundance for each PTEN probe for each cell line. The HT-144 PTEN gene lacks the exon 3 sequence which is reflected in the reduced level of RNA detected by the PTEN exon 3-4 probe. COLO829 has exon 6 deleted from the PTEN locus and the lack of RNA detected by the PTEN exon 6 probe while RNA expression is still detected by all other probes is consistent with this loss. The PTEN gene is truncated in PC-3, with exons 3-9 deleted, and RNA detected in this cell line by the PTEN exon 1-2 probe alone is consistent with this deletion.

FIG. 4 illustrates PTEN expression in normal prostate tissue detected by all 9 PTEN probes, which demonstrates heterogeneity in transcript abundance. Normalized counts representing transcript abundance for each PTEN probe in 9 normal prostate tissues is shown, along with the normalized counts for each probe measured in the NORMAL RNA POOL.

FIG. 5 illustrates representative normal prostate and prostate tumors with PTEN status determined by IHC of INTACT, BORDERLINE, or LOSS (left column) and the Log₂ Ratio profiles for all 9 PTEN probes from each tissue (right column). The scale bar of 500 μm in panel D applies to all 4 tissue images. The horizontal line at the value 0 on all graphs represents the NORMAL RNA POOL consisting of RNA pooled from 11 normal prostate tissues.

FIG. 6 illustrates distribution of 48-case prostate tumor data set by % PTEN LOSS determined by IHC and Mean Log₂ Ratio score. Prostate tumor samples with % PTEN loss of less than 50% as determined by IHC have a protein status of PTEN INTACT (circles), whereas prostate tumor samples with % PTEN loss of greater than or equal to 50% as determined by IHC have a protein status of PTEN LOSS (rectangles). Additionally, prostate tumor samples with 40% to less than 50% loss as determined by IHC have a protein status of BORDERLINE INTACT (white circles) and with from 50% to 60% loss as determined by IHC have a protein status of BORDERLINE LOSS (white rectangles). The cut-off between PTEN LOSS and PTEN INTACT status as determined by the Mean Log₂ Ratio scores from the mRNA expression data for the prostate tumor samples is −0.92, where less than the cut-off predicts LOSS and greater than that predicts INTACT.

FIG. 7 is a comparison of Mean Log2 Ratio scores for 11 prostate tumors where RNA was extracted from tumor tissue and adjacent normal stroma and from tumor tissue scraped to exclude normal prostate tissue. The difference in Mean Log2 Ratio scores between the two methods of preparation for the samples was believed not to be significant.

### DETAILED DESCRIPTION OF THE DISCLOSURE

### I. Abbreviations and Definitions

Unless defined otherwise, technical and scientific terms used herein have the same meaning as commonly understood by a person of ordinary skill in the art.

It should also be understood that, unless clearly indicated to the contrary, in any methods claimed herein that include more than one step or act, the order of the steps or acts of the method is not necessarily limited to the order in which the steps or acts of the method are recited.

As used herein, the singular terms “a,” “an,” and “the” include plural referents unless context clearly indicates otherwise. Similarly, the word “or” is intended to include “and” unless the context clearly indicates otherwise. The term “includes” is defined inclusively, such that “includes A or B” means including A, B, or A and B.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of” or “exactly one of.” “Consisting essentially of,” when used in the claims, shall have its ordinary meaning as used in the field of patent law.

The terms “comprising,” “including,” “having,” and the like are used interchangeably and have the same meaning. Similarly, “comprises,” “includes,” “has,” and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a device having components a, b, and c” means that the device includes at least components a, b, and c. Similarly, the phrase: “a method involving steps a, b, and c” means that the method includes at least steps a, b, and c. Moreover, while the steps and processes may be outlined herein in a particular order, the skilled artisan will recognize that the ordering steps and processes may vary.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

As used herein, the term “about” means+/−10% of the identified value, such as +/−5% of the identified value, such as +/−4% of the identified value, such as +/−3% of the identified value, such as +/−2% of the identified value, or such as +/−1% of the identified value.

Cellular sample: As used herein, the term “cellular sample” refers to any sample containing intact cells, such as cell cultures, bodily fluid samples or surgical specimens taken for pathological, histological, or cytological interpretation.

Complementary: As used herein, the term “complementary” refers to a nucleotide sequence that base-pairs by non-covalent bonds to all or a region of a target nucleic acid (e.g., a template RNA or other region of the double stranded product nucleic acid). In the canonical Watson-Crick base pairing, adenine (A) forms a base pair with thymine (T), as does guanine (G) with cytosine (C) in DNA. In RNA, thymine is replaced by uracil (U). As such, A is complementary to T and G is complementary to C. In RNA, A is complementary to U and vice versa. Typically, “complementary” refers to a nucleotide sequence that is at least partially complementary. The term “complementary” may also encompass duplexes that are fully complementary such that every nucleotide in one strand is complementary to every nucleotide in the other strand in corresponding positions. In certain cases, a nucleotide sequence may be partially complementary to a target, in which not all nucleotides are complementary to every nucleotide in the target nucleic acid in all the corresponding positions. For example, a probe may be perfectly (i.e., 100%) complementary to the target nucleic acid, or the probe and the target nucleic acid may share some degree of complementarity which is less than perfect (e.g., 70%, 75%, 85%, 90%, 95%, 99%). The percent identity of two nucleotide sequences can be determined by aligning the sequences for optimal comparison purposes (e.g., gaps can be introduced in the sequence of a first sequence for optimal alignment). The nucleotides at corresponding positions are then compared, and the percent identity between the two sequences is a function of the number of identical positions shared by the sequences (i.e., % identity=# of identical positions/total # of positions×100). When a position in one sequence is occupied by the same nucleotide as the corresponding position in the other sequence, then the molecules are identical at that position. A non-limiting example of such a mathematical algorithm is described in Karlin et al., Proc. Nati. Acad. Sci. USA 90:5873-5877 (1993). Such an algorithm is incorporated into the NBLAST and XBLAST programs (version 2.0) as described in Altschul et al., Nucleic Acids Res. 25:389-3402 (1997). When utilizing BLAST and Gapped BLAST programs, the default parameters of the respective programs (e.g., NBLAST) can be used. In one aspect, parameters for sequence comparison can be set at score=100, wordlength=12, or can be varied (e.g., wordlength=5 or wordlength=20).

Probe: As used herein, the term “probe” shall refer to a nucleic acid molecule having a sequence that is complementary to a target nucleic acid sequence in a sample, such that the probe specifically hybridizes to the target nucleic acid sequence but does not specifically hybridize to non-target sequences in the sample.

RNA sample: As used herein, the term “RNA sample” shall refer to any sample in which RNA has been purified from other constitutes of the sample, such as proteins, DNA, lipids, etc.

Sample: As used herein, the terms “sample,” “biological sample” or “specimen” or the like refers to any sample including a biomolecule (such as a protein, a peptide, a nucleic acid, a lipid, a carbohydrate, or a combination thereof) that is obtained from any organism including viruses. Other examples of organisms include mammals (such as humans; veterinary animals like cats, dogs, horses, cattle, and swine; and laboratory animals like mice, rats, and primates), insects, annelids, arachnids, marsupials, reptiles, amphibians, bacteria, and fungi. Biological samples include tissue samples (such as tissue sections and needle biopsies of tissue), cell samples (such as cytological smears such as Pap smears or blood smears or samples of cells obtained by microdissection), or cell fractions, fragments, or organelles (such as obtained by lysing cells and separating their components by centrifugation or otherwise). Other examples of biological samples include blood, serum, urine, semen, fecal matter, cerebrospinal fluid, interstitial fluid, mucous, tears, sweat, pus, biopsied tissue (for example, obtained by a surgical biopsy or a needle biopsy), nipple aspirates, cerumen, milk, vaginal fluid, saliva, swabs (such as buccal swabs), or any material containing biomolecules that is derived from a first biological sample. In certain embodiments, the term “biological sample” as used herein refers to a sample (such as a homogenized or liquefied sample) prepared from a tumor or a portion thereof obtained from a subject.

Section: When used as a noun, a thin slice of a tissue sample suitable for microscopic analysis, typically cut using a microtome. When used as a verb, the process of generating a section.

Serial section: As used herein, the term “serial section” shall refer to any one of a series of sections cut in sequence by a microtome from a tissue sample. For two sections to be considered “serial sections” of one another, they do not necessarily need to be consecutive sections from the tissue, but they should generally contain sufficiently similar tissue structures in the same spatial relationship, such that the structures can be matched to one another after histological staining.

Stain: When used as a noun, the term “stain” shall refer to any substance that can be used to visualize specific molecules or structures in a cellular sample for microscopic analysis, including brightfield microscopy, fluorescent microscopy, electron microscopy, and the like. When used as a verb, the term “stain” shall refer to any process that results in deposition of a stain on a cellular sample.

Subject: As used herein, the term “subject” or “individual” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human.

Test sample: A sample obtained from a subject having an unknown status for an mRNA of interest at the time the sample is obtained.

Tissue sample: As used herein, the term “tissue sample” shall refer to a cellular sample that preserves the cross-sectional spatial relationship between the cells as they existed within the subject from which the sample was obtained.

Tumor: As used herein, the term “tumor” refers to all neoplastic cell growth and proliferation, whether malignant or benign, and all pre-cancerous and cancerous cells and tissues. In some embodiments, the tumor is a malignant cancerous tumor (i.e., cancer). In some embodiments, the tumor is a solid tumor or a non-solid or soft tissue tumor. Examples of soft tissue tumors include leukemia (e.g., chronic myelogenous leukemia, acute myelogenous leukemia, adult acute lymphoblastic leukemia, acute myelogenous leukemia, mature B-cell acute lymphoblastic leukemia, chronic lymphocytic leukemia, prolymphocytic leukemia, or hairy cell leukemia) or lymphoma (e.g., non-Hodgkin's lymphoma, cutaneous T-cell lymphoma, or Hodgkin's disease). A solid tumor includes any cancer of body tissues other than blood, bone marrow, or the lymphatic system. Solid tumors can be further divided into those of epithelial cell origin and those of non-epithelial cell origin. Examples of epithelial cell solid tumors include tumors of the gastrointestinal tract, colon, colorectal (e.g., basaloid colorectal carcinoma), breast, prostate, lung, kidney, liver, pancreas, ovary (e.g., endometrioid ovarian carcinoma), head and neck, oral cavity, stomach, duodenum, small intestine, large intestine, anus, gall bladder, labium, nasopharynx, skin, uterus, male genital organ, urinary organs (e.g., urothelium carcinoma, dysplastic urothelium carcinoma, transitional cell carcinoma), bladder, and skin. Solid tumors of non-epithelial origin include sarcomas, brain tumors, and bone tumors.

Tumor sample: As used herein, the terms “tumor sample” or “tumor tissue” encompass samples prepared from a tumor or from a sample potentially including or suspected of comprising cancer cells, or to be tested for the potential presence of cancer cells, such as a lymph node. As used herein, the term “tumor” refers to a mass or a neoplasm, which itself is defined as an abnormal new growth of cells that usually grow more rapidly than normal cells and will continue to grow if not treated sometimes resulting in damage to adjacent structures. Tumor sizes can vary widely. A tumor may be solid, or fluid filled. A tumor can refer to benign (not malignant, generally harmless), or malignant (capable of metastasis) growths. Some tumors can contain neoplastic cells that are benign (such as carcinoma in situ) and, simultaneously, contain malignant cancer cells (such as adenocarcinoma). This should be understood to include neoplasms located in multiple locations throughout the body. Therefore, for purposes of the disclosure, tumors include primary tumors, lymph nodes, lymphatic tissue, and metastatic tumors.

II. Methods of Evaluating mRNA Expression

Disclosed herein are methods of analyzing mRNA expression in a sample by quantifying one or more distinct portions of the same mRNA transcript.

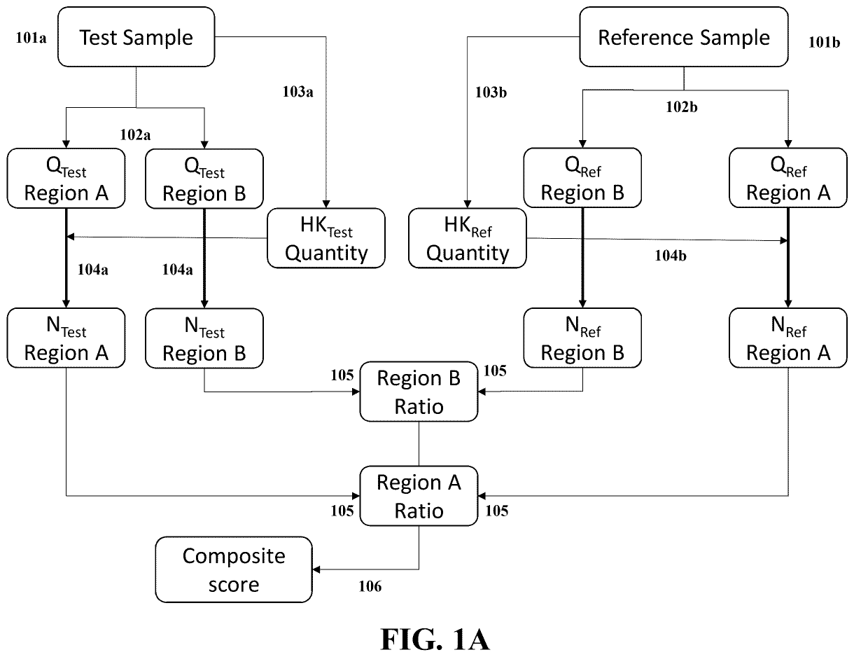

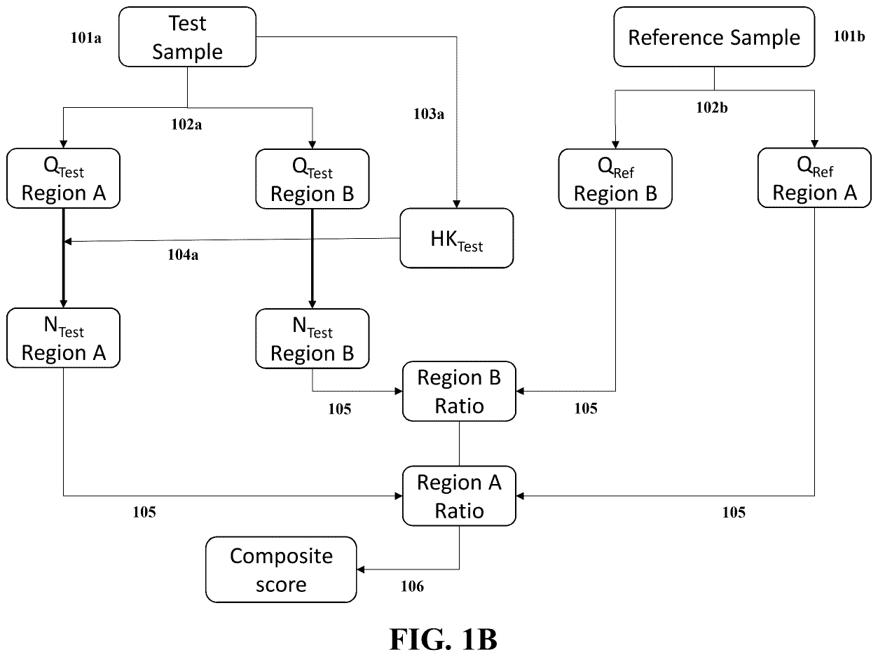

Non-limiting workflows are set forth at FIG. 1A and FIG. 1B. As illustrated therein, a test sample 101a and a reference sample 101b are obtained. The test sample 101a is evaluated to determine a quantity of each distinct region of the target mRNA (Q_{Test}) 102a and one or more housekeeping mRNA (HK_{Test}) 103a. Each individual Q_{Test} is then normalized against HK_{Test} to obtain a normalized quantity for each region of the mRNA (N_{Test}) 104a. The reference sample is also evaluated to determine a quantity of each distinct region of the target mRNA 102b (Q_{Ref}) and, optionally the housekeeping mRNA (HK_{Ref}). In some embodiments (such as that illustrated at FIG. 1A), Q_{Ref} is normalized against HK_{Ref} 104b to obtain a normalized quantity for each region of the mRNA (N_{Ref}) and a ratio between N_{Test} and N_{Ref} is generated 105. In other embodiments (such as that illustrated at FIG. 1B), the reference sample 101b includes a known quantity of the mRNA transcript, in which case a ratio between N_{Test} and Q_{Ref} is generated 105. The ratios are then applied to a trained classifier to generate a composite score indicative of the status of the mRNA in the test sample 106.

IIA. mRNA of Interest

The individual regions of the mRNA may be, for example, individual exons, one or more consecutive exons (for example, a single quantity accounting for adjacent exons within the same transcript), and/or a UTR region (including a 5′ UTR or a 3′UTR). In some embodiments, a quantity is determined for at least one exon and at least one UTR of the mRNA. In another embodiment, a quantity is determined for at least 2 exons of the mRNA. In another embodiment, a quantity is determined for at least 2 exons and at least one UTR of the mRNA. In another embodiment, a quantity is determined for at least 2 exons but less than the full set of exons of the mRNA. In another embodiment, a quantity is determined for at least 2 exons but less than the full set of exons and at least one UTR of the mRNA.

Exemplary mRNA targets of interest include a human PTEN mRNA, a human MYC mRNA, a human TROP2 mRNA, a human CRBN mRNA, a human AR mRNA, a human AXL mRNA, a human DLL3 mRNA, a human FGFR2IIIb mRNA, a human FOLR1 mRNA, a human HER2 mRNA, a human LAG3 mRNA, a human MET mRNA, a human TIGIT mRNA, and a human TP53 mRNA.

In a specific embodiment, the mRNA is a PTEN mRNA and the regions are selected from the group consisting of exon 1, exon 2, a region spanning exons 1 and 2 (exon 1-2), exon 3, exon 4, a region spanning exons 3 and 4 (exon 3-4), exon 5, exon 6, exon 7, exon 8, exon 9, and one or more regions of a 3′ UTR. In another specific embodiment, the mRNA regions have a length of up to about 500 nucleotides and comprise, consist essentially, or consist of SEQ ID NO: 1-9. In another specific embodiment, the mRNA regions are exon 7 and exon 9. In another specific embodiment, the mRNA regions are exon 1-2, exon 6, exon 7, and exon 9. In another specific embodiment, the mRNA regions are exon 5, exon 6, exon 7, and exon 9. In another specific embodiment, the mRNA regions are exon 7, exon 9, and a 3′ UTR region. In another specific embodiment, the mRNA regions are exon 5, exon 7, and exon 9. In another specific embodiment, the mRNA regions have a length of up to 500 nucleotides and comprise, consist essentially, or consist of SEQ ID NO: 5 and SEQ ID NO: 7. In another specific embodiment, the mRNA regions have a length of from about 50 to about 500 nucleotides and comprise, consist essentially, or consist of SEQ ID NO: 1, SEQ ID NO: 4, SEQ ID NO: 5, and SEQ ID NO: 7. In another specific embodiment, the mRNA regions have a length of from up to about 500 nucleotides and comprise, consist essentially, or consist of SEQ ID NO: 3, SEQ ID NO: 4, SEQ ID NO: 5, and SEQ ID NO: 6. In another specific embodiment, the mRNA regions have a length of from up to about 500 nucleotides and comprise, consist essentially, or consist of SEQ ID NO: 5, SEQ ID NO: 7, and SEQ ID NO: 8. In another specific embodiment, the mRNA regions have a length of up to about 500 nucleotides and comprise, consist essentially, or consist of SEQ ID NO: 3, SEQ ID NO: 5, and SEQ ID NO: 7. PTEN mRNA sequences can be found at GenBank accession number NM_000314 (available at ncbi.nlm.nih.gov, last accessed 25 Jan. 2023).

IIB. Housekeeping mRNA

The housekeeping mRNA are used to normalize the quantities of the distinct regions of the target mRNA. Methods identifying gene panels for the normalization of mRNA expression counts are well known in the art. See, for example, Cheng, Chervoneva, Dheda, Eisenberg & Levanon, Jo, Krasnov, Nanostring Technical Note, Sharan, Silvia, and Zhu. Any mRNA that is constitutively and stably expressed in the tissue type from which the test sample is derived may be used as a housekeeping mRNA.

In an embodiment, more than one (including 2, 3, 4, 5, 6, 7, 8, 9, 10, 11, 12 or more) housekeeping mRNAs are used, wherein each housekeeping mRNA is constitutively and stably expressed in the tissue type from which the test sample is derived. In a further embodiment, at least 3 housekeeping mRNA (including 3, 4, 5, 6, 7, 8, 9, 10, 11, 12 or more) are used, wherein each housekeeping mRNA is constitutively and stably expressed in the tissue type from which the test sample is derived and at least 1 has a high expression level, at least 1 has a medium expression level, and at least 1 has a low expression level. In this context, a housekeeping mRNA having: (a) a “high expression level” is one having an average expression level that places it in the highest tertile of genes expressed in the tissue type from which the test sample is derived; (b) a “medium expression level” is one having an average expression level that places it in the middle tertile of genes expressed in the tissue type from which the test sample is derived; and (c) a “low expression level” is one having an average expression level that places it in the lower tertile of genes expressed in the tissue type from which the test sample is derived.

In a specific embodiment, the test sample is a prostate sample, the mRNA of interest is a PTEN mRNA, and the housekeeping mRNA comprise 1, 2, 3, 4, 5, 6, 7, 8, 9, and each of the genes selected from the group consisting of GPATCH3, TBP, PUM1, NRDE2, ARMH3, TMUB2, UBB, RPL19, RPLP0, and MRPS5.

### IIC. Test Sample

The test sample 101a is derived from a subject having or suspected of having a disease state in which the status of the mRNA of interest is unknown. The sample can be any sample type that is compatible with the methodology used for evaluating the mRNA. As an example, the test sample is derived from a tumor suspected of being or diagnosed as a being a cancer, such as a carcinoma, sarcoma, leukemia, or lymphoma, including but not limited to tumors of the breast, skin, liver, pancreas, gall bladder, lung, urinary system, colorectal tract, stomach, small intestines, prostate, cervix uteri, uterus, ovaries, fallopian tubes. In another example, the test sample is an RNA sample derived from a tumor suspected of being or diagnosed as a being a cancer, such as a carcinoma, sarcoma, leukemia, or lymphoma, including but not limited to tumors of the breast, skin, liver, pancreas, gall bladder, lung, urinary system, colorectal tract, stomach, small intestines, prostate, cervix uteri, uterus, ovaries, fallopian tubes. In another example, the test sample is derived from a formalin fixed paraffin embedded tissue sample of a tumor suspected of being or diagnosed as a being a cancer, such as a carcinoma, sarcoma, leukemia, or lymphoma, including but not limited to tumors of the breast, skin, liver, pancreas, gall bladder, lung, urinary system, colorectal tract, stomach, small intestines, prostate, cervix uteri, uterus, ovaries, fallopian tubes. In another embodiment, the test sample is a peripheral blood sample or cell free nucleic acid sample derived from a subject during or after treatment for a cancer, such as a carcinoma, sarcoma, leukemia, or lymphoma, including but not limited to tumors of the breast, skin, liver, pancreas, gall bladder, lung, urinary system, colorectal tract, stomach, small intestines, prostate, cervix uteri, uterus, ovaries, fallopian tubes. In a specific embodiment, the mRNA of interest is a PTEN mRNA, and the test sample is an RNA sample derived from a prostate tumor.

### IID. Reference Sample

The reference sample 101b may be (a) a sample derived from one or more samples having a known status for the mRNA of interest, (b) a sample derived from one or more normal samples corresponding to the same type of sample from which the test sample is derived, or (c) contains a known quantity of the mRNA of interest.

As used herein, a sample has a “known status for the mRNA of interest” when it has already been determined to either lack or contain the mRNA of interest, either by detection of the mRNA itself or by detection of a polypeptide encoded by the mRNA. The reference sample may be generated from 1 or more (including 2, 3, 4, 5, 6, 7, 8, 9, 10 or more) distinct samples having the known status for the mRNA of interest. At least one of the samples used to generate the reference sample should be known to express the mRNA of interest. Where multiple samples are used to generate the reference sample, the reference sample may comprise a mix of samples having different expression levels (for example, for example, expressers and non-expressers). In a specific example, the reference sample comprises equal amounts of sample having different expression levels. In a specific example, the test sample is an RNA sample derived from a tumor sample and the reference sample an RNA sample 1 or more (including 2, 3, 4, 5, 6, 7, 8, 9, 10 or more) tumor samples of the same type as the test sample. In another specific example, the test sample is an RNA sample derived from a tumor sample and the reference sample is an RNA sample comprising equimolar amounts of RNA derived from high expressing tumor(s) (i.e. tumors having an expression level of the mRNA of interest that is greater than about 50% of a cohort of tumors tested) and low expressing tumor(s) (i.e. tumors having an expression level less than about 50% of a cohort of tumors tested). In a specific example, the mRNA of interest is a PTEN mRNA, the test sample is an RNA sample derived from a prostate tumor, and the reference sample is an RNA sample derived from 1 or more (including 2, 3, 4, 5, 6, 7, 8, 9, 10 or more) prostate tumor samples having a known status for the PTEN mRNA. In another specific example, the mRNA of interest is a PTEN mRNA, the test sample is an RNA sample derived from a prostate tumor, and the reference sample is an RNA sample comprising equimolar amounts of RNA from 2, 3, 4, 5, 6, 7, 8, 9, 10 or more prostate tumor samples wherein at least some of the prostate tumors PTEN expressers and some of the prostate tumors are known PTEN non-expressers. In another specific example, the mRNA of interest is a PTEN mRNA, the test sample is an RNA sample derived from a prostate tumor, and the reference sample is an RNA sample comprising equimolar amounts of RNA from PTEN-expressing prostate tumor(s) and PTEN non-expressing prostate tumor(s). In another specific example, the mRNA of interest is a PTEN mRNA, the test sample is an RNA sample derived from a prostate tumor, and the reference sample is an RNA sample comprising equimolar amounts of RNA from PTEN-high-expressing prostate tumor(s) and PTEN-low-expressing prostate tumor(s). As used herein a “PTEN-high-expressing prostate tumor” is one that has an expression level of PTEN mRNA or protein that is greater than 50% of a cohort of prostate tumors tested, and a “PTEN-low-expressing prostate tumor” is one that has an expression level of PTEN mRNA or protein that is less than about 50% of a cohort of prostate tumors tested. In another specific example, the mRNA of interest is a PTEN mRNA, the test sample is a peripheral blood sample or cell free nucleic acid sample derived from a subject during or after treatment for prostate cancer or an early stage prostate tumor, and the reference sample is an RNA sample derived from 2, 3, 4, 5, 6, 7, 8, 9, 10 or more peripheral blood sample or cell free nucleic acid samples known to contain a PTEN mRNA.

As used herein, a sample is a “normal sample” when it has the same organ and tissue origin as the test sample but is not diseased (for example, where the test sample is an epithelial prostate tumor, the “normal sample” is normal prostate epithelium). The reference sample may be generated from 1 or more (including 2, 3, 4, 5, 6, 7, 8, 9, 10 or more) distinct normal samples. In a specific example, the reference sample is an RNA sample comprising equimolar amounts of 2, 3, 4, 5, 6, 7, 8, 9, 10 or more distinct normal samples. In a specific example, the test sample is an RNA sample derived from a tumor sample and the reference sample an RNA sample derived from 1 or more (including 2, 3, 4, 5, 6, 7, 8, 9, 10 or more) normal samples of the tissue type from the which tumor is derived. In another specific example, the mRNA of interest is a PTEN mRNA, the test sample is an RNA sample derived from a prostate tumor sample, and the reference sample is an RNA sample comprising equimolar amounts of RNA derived from 2, 3, 4, 5, 6, 7, 8, 9, 10 or more normal prostate samples. In another specific example, the mRNA of interest is a PTEN mRNA, the test sample is a peripheral blood sample or cell free nucleic acid sample derived from a subject during or after treatment for prostate cancer or an early stage prostate tumor, and the reference sample is an RNA sample derived peripheral blood samples or cell free nucleic acid samples from 2, 3, 4, 5, 6, 7, 8, 9, 10 or more distinct male subjects that do not have prostate cancer.

As used herein, a sample has a “known quantity of the mRNA of interest” when the molar concentration of the mRNA of interest is known. Such an example can be generated, for example, by in vitro transcribing the mRNA of interest from a DNA vector, isolating the transcribed mRNA, and diluting the isolated mRNA to a known RNA concentration in a buffer that is compatible with the selected mRNA evaluation method.

II.E. Quantification of Q_{Test}, HK_{Test}, Q_{Ref}, and HK_{Ref}

A quantity of each distinct region of the target mRNA is determined in the test sample (Q_{Test}) 102a and in the reference sample 102b (Q_{Ref}). Additionally, a quantity of one or more (including 2, 3, 4, 5, 6, or more) housekeeping mRNA is determined in the test sample 103a (HK_{Test}) and, optionally, in the reference sample 103b (HK_{Ref}). Quantities of Q_{Test}, Q_{Ref}, HK_{Test}, and HK_{Ref} is determined by a quantitative or semi-quantitative RNA detection method. In an example, the RNA detection method is an amplification-based method, such as quantitative reverse transcription PCR (qRT-PCR) and digital PCR. In other examples, the RNA detection method is a non-amplification-based method of quantifying mRNA, such as, for example, branched chain nucleic acid hybridization reactions (reviewed by Brunstein), colloidal gold-labeled hybridization probes (reviewed by Brunstein), and reactions using “barcoded” nucleic acid hybridization probes (such as those disclosed in Geiss, W O 2007-076128 A2 and WO 2010-019826 A1, the disclosure of which is hereby incorporated by reference herein in its entirety.

In a specific example, a non-amplification-based method is used comprising a pair of sequence-specific probe pairs: (A) a capture probe comprising (a1) a nucleic acid sequence complementary to a first sequence in the target mRNA and (a2) a capture moiety; and (B) a reporter probe comprising: (b1) a nucleic acid sequence complementary to a second sequence of the target mRNA, wherein the first sequence and the second sequence are disposed in the same target region of the mRNA and do not overlap; and (b2) a detectably-labeled reporter region, such as a fluorescent label. A separate probe pair is provided for each distinct mRNA region to be evaluated, as well as a probe pair for each housekeeping mRNA. Each separate probe pair has a distinct detectable label. Exemplary detectable labels include fluorophores, radioactive isotopes, colloidal gold nanoparticles, and quantum dots. Specific example of probe pair structures and associated labels are disclosed at, for example, Geiss, W O 2007-076128 A2 and WO 2010-019826 A1 (the disclosures of which are hereby incorporated by reference herein their entireties).

The probe pairs are contacted with an RNA sample under conditions that permit specific hybridization of the probe pairs to their intended targets to form a tripartite structure of capture probe and reporter probe hybridized to the target mRNA. Unhybridized probes are removed, and the tripartite structures are immobilized to a solid support via specific binding between the capture moiety and a capture binder on the solid surface. Exemplary capture moiety capture binder pairs include biotin: biotin binding molecules (such as streptavidin and streptavidin derivatives), hapten: anti-hapten antibodies, and epitope tag: anti-epitope tag antibodies. The detectable label of the immobilized tripartite structures is then identified and counted. For example, where the detectable label is a fluorescent molecule, the solid support may be scanned with a fluorescent microscope, such as is used in the commercially available NANOSTRING NCOUNTER analysis system (Nanostring Technologies, Inc.). In a specific embodiment, at least 2 probe pairs as described in this paragraph are selected that target regions of a PTEN mRNA, wherein the regions are selected from the group consisting of exon 1, exon 2, a region spanning exons 1 and 2 (exon 1-2), exon 3, exon 4, a region spanning exons 3 and 4 (exon 3-4), exon 5, exon 6, exon 7, exon 8, exon 9, and one or more regions of a 3′ UTR. In another specific embodiment, the probe pairs target a sequence having a length of from 50 to 500 nucleotides and comprising, consisting essentially of, or consisting of SEQ ID NO: 1-9. In another specific embodiment, a first probe pair is provided targeting PTEN exon 7, and a second probe pair is provided targeting PTEN exon 9. In another specific embodiment, a first probe pair is provided targeting PTEN exon 1-2, a second probe pair is provided targeting PTEN exon 6, a third probe pair targeting PTEN exon 7, and a fourth probe pair is provided targeting PTEN exon 9. In another specific embodiment, a first probe pair is provided targeting PTEN exon 5, a second probe pair is provided targeting PTEN exon 6, a third probe pair is provided targeting PTEN exon 7, and a fourth probe pair is provided targeting PTEN exon 9. In another specific embodiment, a first probe pair is provided targeting PTEN exon 7, a second probe pair is provided targeting PTEN exon 9, and a third probe pair is provided targeting 3′ UTR region. In another specific embodiment, a first probe pair is provided targeting PTEN exon 5, a second probe pair is provided targeting PTEN exon 7, and a third probe pair is provided targeting PTEN exon 9. In another specific embodiment, a first probe pair is provided targeting SEQ ID NO: 5 and a second probe pair is provided targeting SEQ ID NO: 7. In another specific embodiment, a first probe pair is provided targeting SEQ ID NO: 1, a second probe pair is provided targeting SEQ ID NO: 4, a third probe pair is provided targeting SEQ ID NO: 5, and a fourth probe pair is provided targeting SEQ ID NO: 7. In another specific embodiment, a first probe pair is provided targeting SEQ ID NO: 3, a second probe pair is provided targeting SEQ ID NO: 4, a third probe pair is provided targeting SEQ ID NO: 5, and a fourth probe pair is provided targeting SEQ ID NO: 6. In another specific embodiment, a first probe pair is provided targeting SEQ ID NO: 5, a second probe pair is provided targeting SEQ ID NO: 7, and a third probe pair is provided targeting SEQ ID NO: 8. In another specific embodiment, a first probe pair is provided targeting SEQ ID NO: 3, a second probe pair is provided targeting SEQ ID NO: 5, and a third probe pair is provided targeting SEQ ID NO: 7. PTEN mRNA sequences can be found at GenBank accession number NM_000314.

As another example, the non-amplification-based method uses a sequence-specific probe comprising (a) a nucleic acid sequence complementary to the target region of the target mRNA and (b) a signal oligonucleotide separated from the sequence of (a) by a cleavable linker. Exemplary cleavable linkers include photo-cleavable linkers, which may be cleaved by a suitable coherent light source (such as UV light or lasers) or incoherent light source (such as an arc lamp of a light emitting diode (LED)). A separate probe is provided for each distinct mRNA region to be evaluated, as well as a probe for each housekeeping mRNA, with each separate probe having a distinct signal oligonucleotide. Specific examples of probe structures are disclosed at, for example, WO 2017-015099 (the disclosure of which is hereby incorporated by reference herein in its entirety). The probes are contacted with a cellular sample (such as a tissue section or cytological sample) under conditions that permit specific hybridization of the probe pairs to their intended targets. Unhybridized probes are removed, the labeled sample is imaged, and regions of interest (ROIs) are identified. Exemplary ROIs include, for example, tissue types present in the sample (for example, tumor tissue, stroma tissue), individual cells, and/or subcellular structures (such as membrane, cytoplasm, nuclei, and the like). At each ROI, the signal oligonucleotide is released by cleaving the cleavable linker (for example, by illuminating the ROI with an appropriate light source to cleave a photo-cleavable linker), and the signal oligonucleotide is collected, detected, and quantified. In some embodiment, detecting comprises a polymerase reaction, a reverse transcriptase reaction, hybridization to an oligonucleotide microarray, mass spectrometry, hybridization to a fluorescent molecular beacon, a sequencing reaction, or NCOUNTER® Molecular Barcodes. A commercially available systems for performing such analysis include a GEOMX digital spatial profiler and/or a NANOSTRING NCOUNTER analysis system (both from Nanostring Technologies, Inc.). In a specific embodiment, at least 2 probes as described in this paragraph are selected that target regions of a PTEN mRNA, wherein the regions are selected from the group consisting of exon 1, exon 2, a region spanning exon 1 and exon 2 (exon 1-2), exon 3, exon 4, a region spanning exons 3 and 4 (exon 3-4), exon 5, exon 6, exon 7, exon 8, exon 9, and one or more regions of a 3′ UTR. In another specific embodiment, the probes target a sequence having a length of from 50 to 500 nucleotides and comprising, consisting essentially of, or consisting of SEQ ID NO: 1-9. In another specific embodiment, a first probe is provided targeting PTEN exon 7, and a second probe is provided targeting PTEN exon 9. In another specific embodiment, a first probe is provided targeting PTEN exon 1-2, a second probe is provided targeting PTEN exon 6, a third probe pair is provided targeting PTEN exon 7, and a fourth probe is provided targeting PTEN exon 9. In another specific embodiment, a first probe is provided targeting PTEN exon 5, a second probe is provided targeting PTEN exon 6, a third probe is provided targeting PTEN exon 7, and a fourth probe is provided targeting PTEN exon 9. In another specific embodiment, a first probe is provided targeting PTEN exon 7, a second probe is provided targeting PTEN exon 9, and a third probe is provided targeting 3′ UTR region. In another specific embodiment, a first probe is provided targeting PTEN exon 5, a second probe is provided targeting PTEN exon 7, and a third probe is provided targeting PTEN exon 9. In another specific embodiment, a first probe is provided targeting SEQ ID NO: 5 and a second probe is provided targeting SEQ ID NO: 7. In another specific embodiment, a first probe is provided targeting SEQ ID NO: 1, a second probe is provided targeting SEQ ID NO: 4, a third probe is provided targeting SEQ ID NO: 5, and a fourth probe is provided targeting SEQ ID NO: 7. In another specific embodiment, a first probe is provided targeting SEQ ID NO: 3, a second probe is provided targeting SEQ ID NO: 4, a third probe is provided targeting SEQ ID NO: 5, and a fourth probe is provided targeting SEQ ID NO: 6. In another specific embodiment, a first probe is provided targeting SEQ ID NO: 5, a second probe is provided targeting SEQ ID NO: 7, and a third probe is provided targeting SEQ ID NO: 8. In another specific embodiment, a first probe is provided targeting SEQ ID NO: 3, a second probe is provided targeting SEQ ID NO: 5, and a third probe is provided targeting SEQ ID NO: 7. PTEN mRNA sequences can be found at GenBank accession number NM_000314 (available at ncbi.nlm.nih.gov, last accessed 25 Jan. 2023).

In each of the foregoing methods, the output of the quantitative or semi-quantitative evaluation of each portion of the mRNA is reported separately (Q_{Test} and Q_{Ref} for that particular portion of the mRNA of interest or HK_{Test} and HK_{Ref} for the individual housekeeping mRNA). Thus, for example, where 3 distinct regions of the mRNA (e.g. exon 1, exon 2, and 3′ UTR-1) and 3 distinct housekeeping mRNA (HK1, HK2, and HK3) are determined in both the test sample and the reference sample, 12 values will be obtained: (1) Q_{Test-Exon1}; (2) Q_{Test-Exon2}; (3) Q_{Test-UTR1}; (4) Q_{Ref-Exon1}; (5) Q_{Ref-Exon2}; (6) Q_{Ref-UTR1}; (7) HK_{Test-HK1}; (8) HK_{Test-HK2}; (9) HK_{Test-HK3}; (10) HK_{Ref-HK1}; (11) HK_{Ref-HK2}; and (12) HK_{Ref-HK3}. These quantities are then used for subsequent normalization and ratio determination steps

### II.F. Normalization and Ratio Generation

Each individual Q_{Test} is normalized against an HK_{Test} to obtain a normalized quantity for each region of the mRNA (N_{Test}) 104a. Approaches for normalizing test mRNA expression values against housekeeping mRNA expression values are known in the art. See, for example, Bhattacharya, Brumbaugh, Erickson, Jia, Molania, Li, NanoString Technical Note, Waggott, and Wang (the disclosure of which is hereby incorporated by reference herein in its entirety).

The N_{Test} values are then transformed to a ratio with either the corresponding N_{Ref} value (FIG. 1A) or the corresponding Q_{Ref} value (FIG. 1B) 105. While a straight ratio between N_{Test} and N_{Ref} or N_{Test} and Q_{Ref} may be used, it may also be beneficial to use a rescaled ratio, for example, by expressing the ratios on a logarithmic scale (such as a log₂ or a log₁₀ scale). Unless otherwise stated, reference to a “ratio” between N_{Test} and N_{Ref} or N_{Test} and Q_{Ref} shall encompass both straight ratios (for example, N_{Test}/N_{Ref}, N_{Test}/Q_{Ref}, N_{Ref}/N_{Test}, or Q_{Ref}/N_{Test}) and rescaled ratios (for example log₂(N_{Test}/N_{Ref}), log₂(N_{Test}/Q_{Ref}, log₂(N_{Ref}/N_{Test}, log₂(Q_{Ref}/N_{Test}), log₁₀(N_{Test}/N_{Ref}), log₁₀(N_{Test}/Q_{Ref}, log₁₀(N_{Ref}/N_{Test}), or log₁₀(Q_{Ref}/N_{Test})).

In embodiments in which the reference sample is derived from normal samples or samples having a known status for the mRNA of interest, Q_{Ref} is also normalized against HK_{Ref} to obtain a normalized expression value for each region of the mRNA in the reference sample (N_{Ref}) 104b. The same housekeeping mRNA used for normalization in the test sample are used in the reference sample and a composite HK_{Ref} score is obtained in the same manner. The Q_{Ref} value is divided by HK_{Ref} to obtain the N_{Ref} value for that portion of the mRNA of interest. Thus, continuing with the example evaluating 3 distinct regions of the mRNA (e.g. exon 1, exon 2, and 3′ UTR-1) and 3 distinct housekeeping mRNA (HK1, HK2, and HK3) in both the test and the reference samples, the following 6 values would be obtained: (1) N_{Test-Exon1}=Q_{Test-Exon1}/HK_{Test}; (2) N_{Test-Exon2}=Q_{Test-Exon2}/HK_{Test}; (3) N_{Test-UTR1}=Q_{Test-UTR1}/HK_{Test}; (4) N_{Ref-Exon1}=Q_{Ref-Exon1}/HK_{Ref}; (5) N_{Ref-Exon2}=Q_{Ref-Exon2}/HK_{Ref}; and (6) N_{Ref-UTR1}=Q_{Ref-UTR1}/HK_{Ref}.

In examples in which the reference sample 101b contains a known quantity of the mRNA transcript (such as that illustrated at FIG. 1B), no normalization of Q_{Ref} is needed. Thus, for example, where 3 distinct regions of the mRNA are evaluated in both the test and reference samples (e.g. exon 1, exon 2, and 3′ UTR-1) and 3 distinct housekeeping mRNA (HK1, HK2, and HK3) are evaluated in the test sample only, the following 6 region ratios would be obtained::(1) N_{Test-Exon1}=Q_{Test-Exon1}/HK_{Test}; (2) N_{Test-Exon2}=Q_{Test-Exon2}/HK_{Test}; (3) N_{Test-UTR1}=Q_{Test-UTR1}/HK_{Test}; (4) Q_{Ref-Exon1}; (5) Q_{Ref-Exon2}; and (6) Q_{Ref-UTR1}.

In a specific embodiment, the mRNA of interest is a PTEN mRNA, the test sample is an RNA sample derived from a tumor, the reference sample is derived from a plurality of normal tissues corresponding to the tumor, and the following a region ratio is obtained for at least regions of the PTEN mRNA selected from the group consisting of a PTEN exon 1-2 region ratio, a PTEN exon 3-4 region ratio, a PTEN exon 5 region ratio, a PTEN exon 6 region ratio, a PTEN exon 7 region ratio, a PTEN exon 8 region ratio, a PTEN exon 9 region ratio, and a PTEN 3′ UTR region ratio.

### II.F. Trained Classifier

The region ratios are input to a trained classifier to obtain a composite score indicative of an mRNA level of the mRNA of interest and/or a diagnostic status for the test sample 106. The trained classifier is obtained by modeling different combinations of region ratios on a machine learning classifier for their ability to predict the diagnostic status of the sample. In practice, the region ratios that are used in the trained classifier are determined for the test sample and input into the trained classifier, and the output of the trained classifier is the composite score.

Any machine learning classifier useful for correlating data with outcomes may be used. Exemplary classes of machine learning classifiers include, for example, logistic regression classifiers, Bayesian classifiers, nearest neighbor classifiers, decision tree classifiers, and support vector machine classifiers.

In an exemplary embodiment, the composite score is indicative of a status of a protein encoded by the mRNA of interest. In such an embodiment, the classifier is trained against a cohort of samples having a known status for the protein. Exemplary protein statuses include: (a) positive or negative, (b) relative protein levels (such as “high” or “low” expressers), (c) rank of the protein expression level across the cohort, (d) expression level relative to the general population (for example, ranked according to quartile, decile, etc.), and (e) absolute expression level (for example, 1+, 2+, or 3+ immunohistochemical status, percentage of cells having positive immunohistochemical staining, or percentage of cells expressing the protein as determined by flow cytometry). For each sample of the training cohort, the following data points are collected: (a) the protein or mRNA status; and (b) a region ratio for a plurality of different regions of the mRNA including, for example, at least 50%, at least 60%, at least 70%, at least 75%, at least 80%, at least 85%, at least 90%, or 100% of the exons of the mRNA of interest, and/or one or more non-coding regions of the mRNA, such as regions of 5′ and 3′ untranslated regions (UTR). Different combinations of region ratios are modeled for their ability to predict the protein status of the sample and a predictive model with acceptable sensitivity and specificity is selected as the trained classifier.

In another exemplary embodiment, the composite score is indicative of a disease status of the subject from which the sample is derived, for example, whether the subject is diagnosed as having a particular disease, whether a disease that the subject is being treated for is likely to progress, whether a disease that the subject has is likely to respond to a particular treatment, or whether a particular disease that the subject has been treated for is likely to recur. In such an embodiment, the classifier is trained against a cohort of samples from a subject having a known outcome (i.e. having the disease or not having the disease, having a disease that has progressed or not progressed within a particular time period, having a disease that has responded to or not responded to a particular treatment, or having recurrence of the disease or not). For each sample of the training cohort, the following data points are collected: (a) the disease status of the subject; and (b) a region ratio for a plurality of different regions of the mRNA including, for example, at least 50%, at least 60%, at least 70%, at least 75%, at least 80%, at least 85%, at least 90%, or 100% of the exons of the mRNA of interest, and/or one or more non-coding regions of the mRNA, such as regions of 5′ and 3′ untranslated regions (UTR). Different combinations of region ratios are then modeled for their ability to predict the disease status, a predictive model is selected for validation, and the validated model is selected as the trained classifier.

In a specific embodiment, the mRNA of interest is a PTEN mRNA, the test sample is a tumor sample, and the composite score is indicative of PTEN protein expression (i.e. PTEN-intact or PTEN-loss). In such an example, the training cohort is a set of tumor samples and each of the following data points is collected for each sample: (a) the PTEN protein status; and (b) a region ratio for a plurality of different regions of the PTEN mRNA including, for example, at least about 50%, at least about 60%, at least about 70%, at least about 75%, at least about 80%, at least about 85%, at least about 90%, or about 100% of the exons of the PTEN mRNA, and/or one or more non-coding regions of the PTEN mRNA, such as regions of 5′ and 3′ untranslated regions (UTR). In an embodiment, the trained classifier correlates region ratios (which may be straight ratios or log ratios (such as log₂ ratios)) of a plurality of PTEN regions to a PTEN protein status (such as PTEN-intact or PTEN-loss), wherein the plurality of PTEN regions are selected from the group consisting of: a region disposed within exon 1 of PTEN (exon 1 region), a region disposed within exon 2 of PTEN (exon 2 region), a region including portions of both exons 1 and 2 of PTEN (exon 1-2 region), a region disposed within exon 3 of PTEN (exon 3 region), a region disposed within exon 4 of PTEN (exon 4 region), a region including portions of both exons 3 and 4 of PTEN (exon 3-4 region), a region disposed within exon 5 of PTEN (exon 5 region), a region disposed within exon 6 of PTEN (exon 6 region), a region disposed within exon 7 of PTEN (exon 7 region), a region disposed within exon 8 of PTEN (exon 8 region), a region disposed within exon 9 of PTEN (exon 9 region), and a region disposed within a 3′ UTR of PTEN (3′ UTR region). In another embodiment, the plurality of PTEN regions comprise, consist essentially of, or consist of nucleotide sequences selected from the group consisting of SEQ ID NO: 1-9. In another embodiment, the plurality of PTEN regions comprise, consist essentially of, or consists of: (a) an exon 7 region and an exon 9 region; (b) an exon 1-2 region, an exon 6 region, an exon 7 region, and an exon 9 region; (c) an exon 5 region, an exon 6 region, an exon 7 region, and an exon 9 region; (d) an exon 7 region, an exon 9 region, and a PTEN 3′ UTR region; and (e) an exon 5 region, an exon 7 region, and an exon 9 region. In another embodiment, the plurality of PTEN regions comprise, consist essentially of, or consists of: (a) SEQ ID NO: 5 and SEQ ID NO: 7; (b) SEQ ID NO: 1, SEQ ID NO: 4, SEQ ID NO: 5, and SEQ ID NO: 7; (c) SEQ ID NO: 3, SEQ ID NO: 4, SEQ ID NO: 5, and SEQ ID NO: 7; (d) SEQ ID NO: 5, SEQ ID NO: 7, and SEQ ID NO: 8; and (e) SEQ ID NO: 3, SEQ ID NO: 5, and SEQ ID NO: 7.

In another embodiment, a trained decision tree classifier correlates region ratios (which may be straight ratios or log ratios (such as log₂ ratios)) of a plurality of PTEN regions to a PTEN protein status (such as PTEN-intact or PTEN-loss), wherein the plurality of PTEN regions are selected from the group consisting of: a region disposed within exon 1 of PTEN (exon 1 region), a region disposed within exon 2 of PTEN (exon 2 region), a region including portions of both exons 1 and 2 of PTEN (exon 1-2 region), a region disposed within exon 3 of PTEN (exon 3 region), a region disposed within exon 4 of PTEN (exon 4 region), a region including portions of both exons 3 and 4 of PTEN (exon 3-4 region), a region disposed within exon 5 of PTEN (exon 5 region), a region disposed within exon 6 of PTEN (exon 6 region), a region disposed within exon 7 of PTEN (exon 7 region), a region disposed within exon 8 of PTEN (exon 8 region), a region disposed within exon 9 of PTEN (exon 9 region), and a region disposed within a 3′ UTR of PTEN (3′ UTR region). In another embodiment, the plurality of PTEN regions comprise, consist essentially of, or consist of nucleotide sequences selected from the group consisting of SEQ ID NO: 1-9. In another embodiment, the plurality of PTEN regions comprise, consist essentially of, or consists of: (a) an exon 7 region and an exon 9 region; (b) an exon 1-2 region, an exon 6 region, an exon 7 region, and an exon 9 region; (c) an exon 5 region, an exon 6 region, an exon 7 region, and an exon 9 region; (d) an exon 7 region, an exon 9 region, and a PTEN 3′ UTR region; and (e) an exon 5 region, an exon 7 region, and an exon 9 region. In another embodiment, the plurality of PTEN regions comprise, consist essentially of, or consists of: (a) SEQ ID NO: 5 and SEQ ID NO: 7; (b) SEQ ID NO: 1, SEQ ID NO: 4, SEQ ID NO: 5, and SEQ ID NO: 7; (c) SEQ ID NO: 3, SEQ ID NO: 4, SEQ ID NO: 5, and SEQ ID NO: 7; (d) SEQ ID NO: 5, SEQ ID NO: 7, and SEQ ID NO: 8; and (e) SEQ ID NO: 3, SEQ ID NO: 5, and SEQ ID NO: 7.

In another embodiment, a trained supervised or unsupervised classifier correlates region ratios (which may be straight ratios or log ratios (such as log₂ ratios)) of a plurality of PTEN regions to a PTEN protein status (such as PTEN-intact or PTEN-loss), wherein the plurality of PTEN regions are selected from the group consisting of: a region disposed within exon 1 of PTEN (exon 1 region), a region disposed within exon 2 of PTEN (exon 2 region), a region including portions of both exons 1 and 2 of PTEN (exon 1-2 region), a region disposed within exon 3 of PTEN (exon 3 region), a region disposed within exon 4 of PTEN (exon 4 region), a region including portions of both exons 3 and 4 of PTEN (exon 3-4 region), a region disposed within exon 5 of PTEN (exon 5 region), a region disposed within exon 6 of PTEN (exon 6 region), a region disposed within exon 7 of PTEN (exon 7 region), a region disposed within exon 8 of PTEN (exon 8 region), a region disposed within exon 9 of PTEN (exon 9 region), and a region disposed within a 3′ UTR of PTEN (3′ UTR region). In another embodiment, the plurality of PTEN regions comprise, consist essentially of, or consist of nucleotide sequences selected from the group consisting of SEQ ID NO: 1-9. In another embodiment, the plurality of PTEN regions comprise, consist essentially of, or consists of: (a) an exon 7 region and an exon 9 region; (b) an exon 1-2 region, an exon 6 region, an exon 7 region, and an exon 9 region; (c) an exon 5 region, an exon 6 region, an exon 7 region, and an exon 9 region; (d) an exon 7 region, an exon 9 region, and a PTEN 3′ UTR region; and (e) an exon 5 region, an exon 7 region, and an exon 9 region. In another embodiment, the plurality of PTEN regions comprise, consist essentially of, or consists of: (a) SEQ ID NO: 5 and SEQ ID NO: 7; (b) SEQ ID NO: 1, SEQ ID NO: 4, SEQ ID NO: 5, and SEQ ID NO: 7; (c) SEQ ID NO: 3, SEQ ID NO: 4, SEQ ID NO: 5, and SEQ ID NO: 7; (d) SEQ ID NO: 5, SEQ ID NO: 7, and SEQ ID NO: 8; and (e) SEQ ID NO: 3, SEQ ID NO: 5, and SEQ ID NO: 7. In an embodiment, the trained classifier is selected from the group consisting of a meta-estimator (such as ada or extraTrees), a support vector machine (such as svm), a neural network (such as nnet), a logistic regression (such as logreg), a Random Forest (such as randomForest, RRF, or ranger), a gradient boosting model (such as xgboost or glmboost), a penalized regression (such as penalized or plr), a multiple discriminant analysis (such as mda), regularized discriminant analysis (such as rda), a nearest neighbor classifier (such as kknn), a linear discriminant analysis (such as lda), a Gaussian processes classifier (such as gausspr), and a C5.0 decision tree and rule-based model (such as C50).

III. Kits for Evaluating mRNA Expression

Also disclosed herein are kits for analyzing mRNA expression in a sample by quantifying distinct portions of the same mRNA transcript.

In an embodiment, the kits comprise a set of nucleic acid probes the kit comprising a plurality of nucleic acid probes complementary to distinct, non-overlapping target regions of the same target mRNA molecule or target cDNA molecule and, optionally, may further comprise a plurality of nucleic acid probes complementary to distinct, non-overlapping target regions of a housekeeping mRNA molecule or housekeeping cDNA molecule. As used herein, a “target region” if a discrete portion of the target nucleic acid that is less than the entire nucleic acid, to which the nucleic acid probes are complementary and capable of hybridizing. The target regions may be uniquely specific regions, such that when a sample is contacted with the probes under stringent hybridization conditions, the probes bind specifically to the target regions, but do not significantly bind to other regions of the mRNA or cDNA or to other nucleic acids contained in the sample, wherein the target regions of the target and housekeeping mRNA and cDNA are exons, groups of adjacent exons, and/or untranslated regions. Exemplary probes include probes useful for non-amplification-based methods of quantifying mRNA or cDNA, such as, for example, by branched chain nucleic acid hybridization reactions (reviewed by Brunstein), colloidal gold-labeled hybridization probes (reviewed by Brunstein), and reactions using “barcoded” nucleic acid hybridization probes (such as those disclosed in Geiss, W O 2007-076128 A2 and WO 2010-019826 A1).

In a specific example, the kit comprises a sequence-specific probe pairs comprising: (A) a capture probe comprising (a1) a nucleic acid sequence complementary to a first sequence in the target mRNA and (a2) a capture moiety; and (B) a reporter probe comprising: (b1) a nucleic acid sequence complementary to a second sequence of the target mRNA, wherein the first sequence and the second sequence are disposed in the same target region of the mRNA and do not overlap; and (b2) a detectably-labeled reporter region, such as a fluorescent label. A separate probe pair is provided for each distinct mRNA region to be evaluated, as well as a probe pair for each housekeeping mRNA. Each separate probe pair has a distinct detectable label. Exemplary detectable labels include fluorophores, radioactive isotopes, colloidal gold nanoparticles, and quantum dots. Specific example of probe pair structures and associated labels are disclosed at, for example, Geiss, W O 2007-076128 A2 and WO 2010-019826 A1. The kits may further comprise a capture binder on a solid surface. The capture binder is capable of binding to the capture moiety of the probe. Exemplary capture moiety: capture binder pairs include biotin: biotin binding molecules (such as streptavidin and streptavidin derivatives), hapten: anti-hapten antibodies, and epitope tag: anti-epitope tag antibodies. In a specific embodiment, at least 2 probe pairs as described in this paragraph are selected complementary to target regions of a PTEN mRNA or cDNA, wherein the regions are selected from the group consisting of exon 1, exon 2, a region spanning exon 1 and exon 2 (exon 1-2), exon 3, exon 4, a region spanning exons 3 and 4 (exon 3-4), exon 5, exon 6, exon 7, exon 8, exon 9, and one or more regions of a 3′ UTR. In another specific embodiment, the probe pairs target a sequence having a length of from 50 to 500 nucleotides and comprising, consisting essentially of, or consisting of SEQ ID NO: 1-9. In another specific embodiment, a first probe pair is provided targeting PTEN exon 7, and a second probe pair is provided targeting PTEN exon 9. In another specific embodiment, a first probe pair is provided targeting PTEN exon 1-2, a second probe pair is provided targeting PTEN exon 6, a third probe pair targeting PTEN exon 7, and a fourth probe pair is provided targeting PTEN exon 9. In another specific embodiment, a first probe pair is provided targeting PTEN exon 5, a second probe pair is provided targeting PTEN exon 6, a third probe pair is provided targeting PTEN exon 7, and a fourth probe pair is provided targeting PTEN exon 9. In another specific embodiment, a first probe pair is provided targeting PTEN exon 7, a second probe pair is provided targeting PTEN exon 9, and a third probe pair is provided targeting 3′ UTR region. In another specific embodiment, a first probe pair is provided targeting PTEN exon 5, a second probe pair is provided targeting PTEN exon 7, and a third probe pair is provided targeting PTEN exon 9. In another specific embodiment, a first probe pair is provided targeting SEQ ID NO: 5 and a second probe pair is provided targeting SEQ ID NO: 7. In another specific embodiment, a first probe pair is provided targeting SEQ ID NO: 1, a second probe pair is provided targeting SEQ ID NO: 4, a third probe pair is provided targeting SEQ ID NO: 5, and a fourth probe pair is provided targeting SEQ ID NO: 7. In another specific embodiment, a first probe pair is provided targeting SEQ ID NO: 3, a second probe pair is provided targeting SEQ ID NO: 4, a third probe pair is provided targeting SEQ ID NO: 5, and a fourth probe pair is provided targeting SEQ ID NO: 6. In another specific embodiment, a first probe pair is provided targeting SEQ ID NO: 5, a second probe pair is provided targeting SEQ ID NO: 7, and a third probe pair is provided targeting SEQ ID NO: 8. In another specific embodiment, a first probe pair is provided targeting SEQ ID NO: 3, a second probe pair is provided targeting SEQ ID NO: 5, and a third probe pair is provided targeting SEQ ID NO: 7. PTEN mRNA sequences can be found at GenBank accession number NM_000314.

As another example, the kit comprises a plurality of sequence-specific probes comprising (a) a nucleic acid sequence complementary to the target region of the target mRNA and (b) a signal oligonucleotide separated from the sequence of (a) by a cleavable linker. Exemplary cleavable linkers include photo-cleavable linkers, which may be cleaved by a suitable coherent light source (such as UV light or lasers) or incoherent light source (such as an arc lamp of a light emitting diode (LED)). A separate probe is provided for each distinct mRNA region to be evaluated, as well as a probe for each housekeeping mRNA, with each separate probe having a distinct signal oligonucleotide. Specific examples of probe structures are disclosed at, for example, WO 2017-015099. In a specific embodiment, at least 2 probes as described in this paragraph are selected that are complementary to a target region of a PTEN mRNA or cDNA, wherein the regions are selected from the group consisting of exon 1, exon 2, a region spanning exons 1 and 2 (exon 1-2), exon 3, exon 4, a region spanning exons 3 and 4 (exon 3-4), exon 5, exon 6, exon 7, exon 8, exon 9, and one or more regions of a 3′ UTR. In another specific embodiment, the probes target a sequence having a length of from 50 to 500 nucleotides and comprising, consisting essentially of, or consisting of SEQ ID NO: 1-9. In another specific embodiment, a first probe is provided targeting PTEN exon 7, and a second probe is provided targeting PTEN exon 9. In another specific embodiment, a first probe is provided targeting PTEN exon 1-2, a second probe is provided targeting PTEN exon 6, a third probe pair is provided targeting PTEN exon 7, and a fourth probe is provided targeting PTEN exon 9. In another specific embodiment, a first probe is provided targeting PTEN exon 5, a second probe is provided targeting PTEN exon 6, a third probe is provided targeting PTEN exon 7, and a fourth probe is provided targeting PTEN exon 9. In another specific embodiment, a first probe is provided targeting PTEN exon 7, a second probe is provided targeting PTEN exon 9, and a third probe is provided targeting 3′ UTR region. In another specific embodiment, a first probe is provided targeting PTEN exon 5, a second probe is provided targeting PTEN exon 7, and a third probe is provided targeting PTEN exon 9. In another specific embodiment, a first probe is provided targeting SEQ ID NO: 5 and a second probe is provided targeting SEQ ID NO: 7. In another specific embodiment, a first probe is provided targeting SEQ ID NO: 1, a second probe is provided targeting SEQ ID NO: 4, a third probe is provided targeting SEQ ID NO: 5, and a fourth probe is provided targeting SEQ ID NO: 7. In another specific embodiment, a first probe is provided targeting SEQ ID NO: 3, a second probe is provided targeting SEQ ID NO: 4, a third probe is provided targeting SEQ ID NO: 5, and a fourth probe is provided targeting SEQ ID NO: 6. In another specific embodiment, a first probe is provided targeting SEQ ID NO: 5, a second probe is provided targeting SEQ ID NO: 7, and a third probe is provided targeting SEQ ID NO: 8. In another specific embodiment, a first probe is provided targeting SEQ ID NO: 3, a second probe is provided targeting SEQ ID NO: 5, and a third probe is provided targeting SEQ ID NO: 7. PTEN mRNA sequences can be found at GenBank accession number NM_000314 (available at ncbi.nlm.nih.gov, last accessed 25 Jan. 2023).

### IV. Examples

### IV.A. Background

Prostate cancer is the third leading cause of cancer-related mortality following lung and colorectal cancer. Despite several biochemical diagnostic tests and histopathological criteria to classify prostate tumors, predicting clinical outcome and stratifying patients is a challenge due to disease heterogeneity and the absence of association between diagnostic results and disease progression. The prevalent standard of care focuses on histopathological Gleason scores and grading system, clinical stage of the disease ranging from primary tumor to metastatic sites, and Prostate Specific Antigen (PSA) biomarker levels in blood. One of the challenging aspects of prostate cancer is substantial chromosomal rearrangements and the genomic heterogeneity of multifocal tumors presenting a variety of sub-clonal cell populations. In recent years, identifying tumor-derived gene expression markers through genomic, transcriptomic and proteomic methods mark a paradigm shift in the search for detection, prognosis and risk assessment of prostate cancer.

Loss of the chromosome region 10q23 is frequently associated with the occurrence of prostate cancer, and subsequently the gene PTEN (Phosphatase and tensin homolog deleted on chromosome 10) was identified at this location. PTEN is a tumor suppressor gene that antagonizes the PI3K-AKT-mTOR signaling pathway. PTEN is expressed in almost all tissues in the body and functions in the regulation of cell division and proliferation. Its mechanism of action as a tumor suppressor involves dephosphorylation of PIP3 produced by PI3Ks, and this action is mediated through a phosphatase domain. A single mutation in this critical region can lead to partial or null phosphatase activity, which decreases or abolishes PTEN tumor suppressor function. Not surprisingly, mutations and loss of PTEN through germline and somatic mutations, deletions and epigenetic mechanisms are common to a variety of solid tumors. Such reduced or absent PTEN protein expression has been identified by immunohistochemical (IHC) analyses in several cancers, including prostatic adenocarcinoma. Aberrations of the PTEN gene is indeed a key prognostic marker associated with poor clinical outcome in prostate cancer. Deletion, mutation or rearrangement of PTEN gene is present in up to 50% of primary to advanced prostate tumors. Loss of PTEN expression is associated with increased recurrence of disease after prostatectomy. Tumor susceptibility has been shown to increase with reduction in PTEN protein levels and both homozygous and heterozygous loss of PTEN is common which is accompanied by loss of protein expression as detected by immunohistochemistry. While some reports indicate that genomic deletions of PTEN are associated with increased disease aggressiveness as measured by Gleason score, other studies failed to find any correlation between PTEN status and Gleason scores. PTEN deletion subtypes have been classified but there is a lack of clinically significant data correlating the different genomic deletions with adverse pathological functions and disease outcomes. These indicate that detailed studies at the molecular level are required to understand the mechanisms and pathways necessary to establish the association between various types of PTEN loss and disease outcome.

The PTEN gene is transcribed into an 8514 nucleotide long mRNA which is composed of 9 exons and encodes a 403 amino acid protein. Instances have been identified where hypermethylation of the PTEN promoter or mutations within the promoter region suppress transcription resulting in the reduction or loss of PTEN protein. Loss of PTEN protein in tumors is primarily the consequence of mutations within the gene sequence. Deletions that extend into the coding sequence or that eliminate the entire gene locus can result in the loss of PTEN transcripts. In addition, point mutations or micro deletions have been found throughout the coding sequence in multiple cancers, with about 50% of these mutations occurring in exon 5 which encodes a segment of the critical phosphatase domain. In these instances, although the PTEN gene is transcribed, mutations not only alter the stability of the mRNA, but also alter the amino acid sequence of the protein and potentially cause loss of function, introducing nonsense mutations or frame-shift mutations which can result in non-functional truncated proteins. Furthermore, multiple instances have been identified where entire exons or several exons have been lost in tumors and in cell lines derived from tumors, either in the PTEN gene itself due to a deletion or only in the PTEN transcript as a result of mis-splicing arising from point mutations at exon/intron splice junctions. Mutations for exon loss have been found to include each of the 9 exons in the PTEN gene.

This study was designed to explore whether PTEN protein loss in prostate adenocarcinomas could be correlated or associated with the loss or reduction of PTEN transcripts. Experiments were performed using the NANOSTRING NCOUNTER analysis system to measure the expression levels of PTEN mRNA in prostate tumors and compare it to the protein expression levels inferred from IHC staining results. The NANOSTRING NCOUNTER technology employs sequence-specific probes to quantitatively identify unique target sequences within the RNA sample being probed but it is not suitable for detecting single nucleotide or short deletion mutations. It is amenable, though, for investigating the loss of individual exon sequences. Therefore, a strategy was implemented using probes corresponding to single exons or exon pairs for all nine exons and the lengthy 3-prime untranslated region (UTR) throughout the full length of PTEN mRNA. The combined results collected using this PTEN exon probe set to interrogate prostate tumors previously scored for PTEN status by IHC were used to design an algorithm that establishes an optimal cut-off between PTEN Intact and PTEN Loss. This robust algorithm can be utilized to predict PTEN loss by measuring abundance of PTEN transcripts in prostate tumor tissue.

### IV.B. Materials and Methods

### IV.B.1. Normal Human Prostate and Adenocarcinoma Tumor Tissue

Assays were performed on commercially sourced normal prostate and prostate adenocarcinoma samples. Two to four 10-micron sections of prostate (normal or tumor resection) FFPE tissue, the number of sections depending on the size of the tumor when one was present in the tissue, were cut from blocks employing practices to reduce the potential for RNA degradation and cross sample contamination, including the use of RNase-free water and changing blades on the microtome between blocks. The cuts for each case were either collected as curls into sterile 1.5 ml microfuge tubes or were deposited onto RNase-free non-charged glass slides if removal of the non-tumor areas was required by scraping. For those samples where sections were mounted on slides, with an H&E stained slide of the tissue as a guide to identify the tumor area, a razor blade was used to scrape away the non-tumor tissue and to collect the tumor tissue from multiple sections of each tumor into a sterile 1.5 ml microfuge tube for RNA extraction. All surfaces, including the razor blades, were treated with RNASEZAP (Cat no. AM9780, Invitrogen/Thermo Fisher Scientific, Waltham, MA) to remove RNases. The data set with tumor-specific scraped samples (scraped-tumor specific) included 48 cases of prostate tissue of which 25 were adenocarcinoma tumors with an IHC status of INTACT (<40% of tumor cells with PTEN loss), 6 were adenocarcinoma tumors with an IHC status of BORDERLINE (40% to 60% of tumor cells with PTEN loss), and 17 were adenocarcinoma tumors with an IHC status of LOSS (>60% of tumor cells with loss of PTEN staining) where PTEN IHC status was assessed with the VENTANA PTEN (SP218) RxDx Assay. The training sample set of 42 cases was a subset of the 48-case set, which was used to develop a Mean Log₂ Ratio score algorithm and cut-off. The final Mean Log₂ Ratio score algorithm and cut-off was tested with a 21 case test set which were samples collected as curls of whole sections with tumor and adjacent normal tissue (not scraped-whole section), and included nine adenocarcinoma tumors with an IHC status of INTACT (0-20% of tumor cells with PTEN loss), two adenocarcinoma tumors with an IHC status of BORDERLINE (one case with 40% of tumor cells with PTEN Loss and one case with 60% of tumor cells with PTEN Loss), and ten adenocarcinoma tumors with an IHC status of LOSS (90-100% of tumor cells with PTEN loss).

### IV.B.2. Human Tumor-Derived Cell Lines

The cell lines NCI-H460, Calu-3, LNCaP, HT-144, COLO829, and PC-3 were obtained from the American Type Culture Collection (ATCC, Manassas, Virginia). The cell lines were tested to ensure that they were mycoplasma-free upon arrival and were cultured according to protocols recommended by ATCC.

### IV.B.3. IHC Methods

The PTEN IHC assay was developed using a recombinant rabbit monoclonal antibody raised against the carboxy-terminus of the ubiquitously expressed 47-kDa human tumor suppressor protein (VENTANA PTEN (SP218) Rabbit Monoclonal Primary Antibody, Cat. No. 07970200001, Roche). The PTEN (SP218) primary antibody reagent is optimized for use on the BENCHMARK series of automated staining instruments in combination with OPTIVIEW DAB IHC Detection Kit (Roche Tissue Diagnostics, Tucson, AZ) and recommended system-level controls.

A prostate adenocarcinoma case was determined to have a PTEN LOSS status when 50% or greater viable malignant cells exhibited no specific cytoplasmic staining in the presence of weak to strong PTEN staining in normal stromal cells, endothelial cells, and peripheral nerve bundles; otherwise, the case was determined to have the status PTEN INTACT. Cases scored as having loss of PTEN protein in the 40% to 60% range were considered BORDERLINE (personal communication).

### IV.B.4. RNA Extraction

RNA samples were prepared by extracting total RNA from FFPE tumor samples using the QIAGEN RNEASY DSP FFPE Kit (Cat. No. 73604; Qiagen, Germany). To compensate for the heterogeneity in gene expression detected between normal samples, a NORMAL RNA POOL containing equal proportions of RNA extracted from 11 normal prostate tissue cases was prepared as a reference to which expression of PTEN in prostate tumors was compared. Total RNA samples were prepared from cell lines with the QIAGEN RNEASY Mini Kit (Cat. #74104; Qiagen, Germany) using 3-5×10⁶ cells each. The quality of the RNA samples was assessed using an Agilent BIOANALYZER (Agilent Technologies, Santa Clara, CA) and RNA concentrations were determined using a Thermo Scientific NANODROP 2000 (Thermo Fisher Scientific, Pittsburgh, PA).

### IV.B.5. NANOSTRING CodeSet and Probe Design

The NANOSTRING NCOUNTER (NanoString Technologies, Seattle, WA) was used to determine transcript levels of PTEN from the prostate tissue. A CodeSet consisting of sequence-specific probes employed in the NCOUNTER application was prepared. Each target sequence is 100 nucleotides in length and is detected by a pair of 50 nucleotides long primers [50]. The CodeSet included 9 probes specifically designed to detect unique sequences along the PTEN mRNA, primarily corresponding to individual exons (probes PTEN exon 1-2, PTEN exon 3-4, PTEN exon 5, PTEN exon 6, PTEN exon 7, PTEN exon 8, PTEN exon 9, PTEN 3′ UTR-1, and PTEN 3′ UTR-2) to potentially identify loss of distinct exons in prostate tumors. See probe target sequences in Table 1 below:

TABLE 1

| | SEQ | | Accession |
|---|---|---|---|
| | ID | | # |
| Name | NO | Target Sequence | (Position) |
| PTEN | 1 | AGGAGATATCAAGAGGATG | NM_000314.4 |
| Exon | | GATTCGACTTAGACTTGAC | (1071-1170) |
| 1-2 | | CTATATTTATCCAAACATT | |
| | | ATTGCTATGGGATTTCCTG | |
| | | CAGAAAGACTTGAAGGCGT | |
| | | ATACA | |
| PTEN | 2 | TGGATTCAAAGCATAAAAA | NM_000314.4 |
| Exon | | CCATTACAAGATATACAAT | (1201-1300) |
| 3-4 | | CTTTGTGCTGAAAGACATT | |
| | | ATGACACCGCCAAATTTAA | |
| | | TTGCAGAGTTGCACAATAT | |
| | | CCTTT | |
| PTEN | 3 | CATGTTGCAGCAATTCACT | NM_000314.4 |
| Exon | | GTAAAGCTGGAAAGGGACG | (1383-1482) |
| 5 | | AACTGGTGTAATGATATGT | |
| | | GCATATTTATTACATCGGG | |
| | | GCAAATTTTTAAAGGCACA | |
| | | AGAGG | |
| PTEN | 4 | AGTAACTATTCCCAGTCAG | NM_000314.4 |
| Exon | | AGGCGCTATGTGTATTATT | (1526-1625) |
| 6 | | ATAGCTACCTGTTAAAGAA | |
| | | TCATCTGGATTATAGACCA | |
| | | GTGGCACTGTTGTTTCACA | |
| | | AGATG | |
| PTEN | 5 | GATATATTCCTCCAATTCA | NM_000314.4 |
| Exon | | GGACCCACACGACGGGAAG | (1700-1799) |
| 7 | | ACAAGTTCATGTACTTTGA | |
| | | GTTCCCTCAGCCGTTACCT | |
| | | GTGTGTGGTGATATCAAAG | |
| | | TAGAG | |
| PTEN | 6 | ATCGATAGCATTTGCAGTA | NM_000314.4 |
| Exon | | TAGAGCGTGCAGATAATGA | (1929-2028) |
| 8 | | CAAGGAATATCTAGTACTT | |
| | | ACTTTAACAAAAAATGATC | |
| | | TTGACAAAGCAAATAAAGA | |
| | | CAAAG | |
| PTEN | 7 | ATGTTAGTGACAATGAACC | NM_000314.4 |
| Exon | | TGATCATTATAGATATTCT | (2134-2233) |
| 9 | | GACACCACTGACTCTGATC | |
| | | CAGAGAATGAACCTTTTGA | |
| | | TGAAGATCAGCATACACAA | |
| | | ATTAC | |
| PTEN | 8 | ACTAGCTGTGGTCTGACCT | NM_000314.6 |
| 3′ | | AGTTAATTTACAAATACAG | (4001-4100) |
| UTR-1 | | ATTGAATAGGACCTACTAG | |
| | | AGCAGCATTTATAGAGTTT | |
| | | GATGGCAAATAGATTAGGC | |
| | | AGAAC | |
| PTEN | 9 | TTGGATGTGCAGCAGCTTA | NM_000314.4 |
| 3′ | | CATGTCTGAAGTTACTTGA | (4831-4930) |
| UTR-2 | | AGGCATCACTTTTAAGAAA | |
| | | GCTTACAGTTGGGCCCTGT | |
| | | ACCATCCCAAGTCCTTTGT | |
| | | AGCTC | |

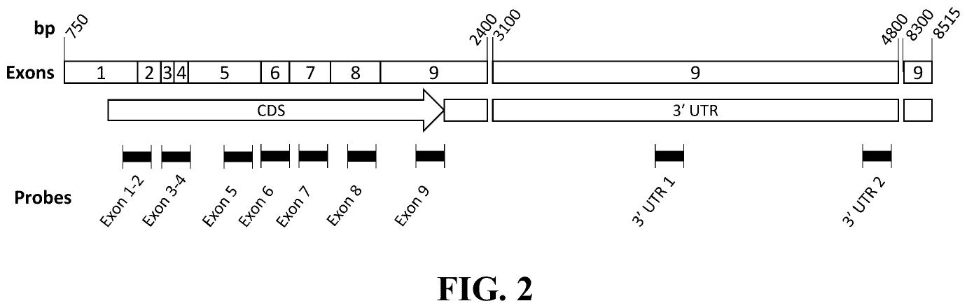

A map of the PTEN mRNA (NCBI Reference Sequence NM_000314.8), location of the PTEN coding sequence within the mRNA and placement of the 9 probes that detect unique sequences within the PTEN transcript are shown in FIG. 2. The probes detecting 2 adjacent exons were necessitated because exons 2, 3, and 4 are each under 100 nucleotides in length. The PTEN exon 9 probe detects a sequence within the coding sequence of exon 9, whereas probes PTEN 3′ UTR-1 and PTEN 3′ UTR-2 correspond to two distinct sites within the long 3-prime untranslated region of exon 9. Probes specific for 10 housekeeping/reference mRNA (GPATCH3, TBP, PUM1, NRDE2, ARMH3, TMUB2, UBB, RPL19, RPLP0, and MRPS5) were also in the CodeSet and were used for normalization of raw counts across the RNA samples tested in the experimental runs.

### IV.B.6. NANOSTRING NCOUNTER Assay

The CodeSet probe mixture was combined with 150 ng RNA for the cell line samples and 300 ng RNA for each FFPE-derived case and the reactions were incubated on a BIORAD T100 Thermal Cycler (BioRad Laboratories, Hercules, CA) at 65° C. for 18 hours. The samples were processed to remove unbound probes on the NCOUNTER Prep Station and raw counts for each probe pair were tabulated on the NCOUNTER Digital Analyzer following the protocol for the NCOUNTER Analysis System provided by the manufacturer. The RNA samples for the 21 case (test sample) and 48 case (training sample) prostate tumor sets were processed over multiple runs of the NANOSTRING NCOUNTER (each run accommodating up to 12 samples) along with a NORMAL RNA POOL sample in each run. The NORMAL RNA POOL was used as a reference to represent the PTEN mRNA level in normal prostate tissue when determining the relative loss of PTEN mRNA in test samples. The raw count output was normalized across all samples using the housekeeping/reference gene data measured in each run with NANOSTRING NSOLVER 4.0 Analysis Software. A score was devised (“Log₂ Ratio”) to assess the relative expression detected by each PTEN exon probe in the prostate tumors by calculating the Log₂ of the ratio of the normalized counts for each probe of a tissue sample to the normalized counts for that probe from the NORMAL RNA POOL (Log₂ Ratio=Log₂ [normalized tissue counts/normalized NORMAL RNA POOL counts]). This metric was used to assess the correlation between PTEN mRNA expression determined by the NANOSTRING assay and PTEN protein expression determined by IHC for each tumor.

### IV.B.7. Algorithm and Cut-Off Development

Several methods were investigated to develop an algorithm and cut-off to optimally differentiate PTEN Intact from PTEN Loss using PTEN expression data from prostate tumor RNA. Initially, the 42-case training sample set was used. Each PTEN exon probe exhibited considerable overlap in Log₂ Ratio scores for cases previously scored as PTEN INTACT and PTEN LOSS by IHC, with scores for the PTEN exon 9 probe showing the smallest range of overlap. Therefore, since single variables may not be good differentiators and would not be good predictors of PTEN loss, machine learning classification models were tested.

Among 20 machine learning classifiers, including ada, svm, nnet, logreg, randomForest, xgboost, extra Trees, penalized, plr, mda, rda, kknn, RRF, lda, glmboost, gausspr, C50, ranger, glmnet, gamboost where simulations were performed using the R package mlr, the classifier “extraTrees” was the best performer with the lowest average log loss and mean classification error based on leave-one-out cross-validation (LOOCV) from 100 bootstrap datasets. Once “extraTrees” was selected, there were two turning parameters applied: number of randomly selected predictors (3 to 9), and number of random cuts (1 to 10). The classifier “extraTrees” produced a maximum accuracy of 81% for 3 random cuts and 6 or 9 randomly selected predictors. However, because this model is complicated and less intuitive, ROC (receiver operating characteristic) analyses were performed. ROC curve analyses, commonly performed to evaluate diagnostic tests, were initially conducted using the mean of the Log₂ Ratio scores for all 9 PTEN probes or for the 4 probes for PTEN exon 6 to PTEN exon 9 to determine an optimal cut-off value from the Youden Index. The Youden Index (J) (J=sensitivity+specificity −1) indicates performance at a given cut-off and the optimal cut-off corresponds to the maximum Youden Index. The performance was not satisfactory by either of these two approaches, so further ROC analyses were conducted for all the combinations of two or more PTEN probes, with the PTEN exon 9 probe always included. This was performed for two scenarios that did not employ bootstrapping: the mean signal of Log₂ Ratio scores for the exon probes included and the first principal component of exon probes. For each approach the optimal cut-off was determined by maximizing the Youden index and the accuracy was calculated. Of the top five models generated by each scenario, four models were in common between the two approaches. Since the mean signal of exon probes is easy to implement and understand, this algorithm and cut-off were selected to evaluate the prediction performance of PTEN expression data with the NANOSTRING NCOUNTER and the final PTEN mRNA algorithm and cut-off.

### IV.C. Results

### IV.C.1. Validation of PTEN Exon-Selective Probes by NANOSTRING

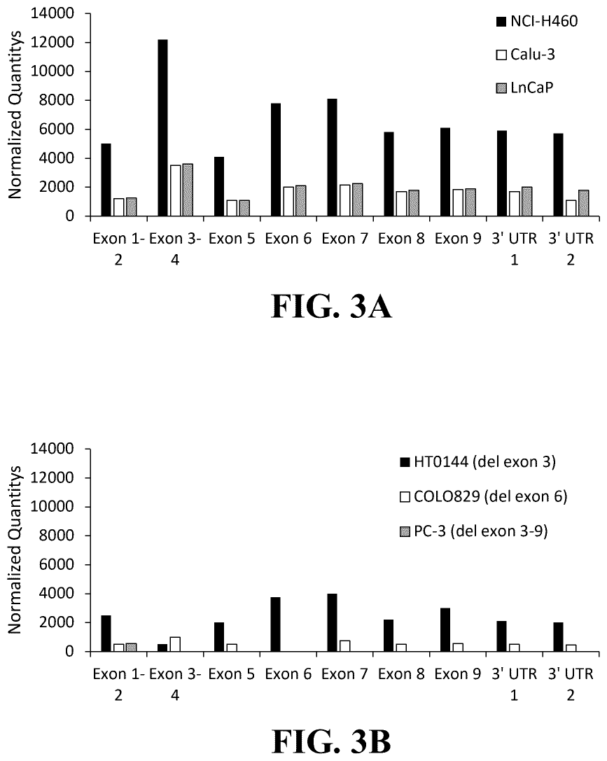

To validate the functionality of each PTEN probe, the probe set was tested with RNA extracted from 3 cell lines; 2 cell lines originating from lung adenocarcinomas, NCI-H460 (high PTEN protein levels) and Calu-3 (low protein levels), and LNCaP, a prostate carcinoma cell line with no PTEN protein expression due to a frameshift mutation in the gene creating a premature stop codon. The normalized transcript counts from these 3 cell lines for each PTEN probe are shown in FIG. 3A. Note that since each probe was a unique sequence with individual binding characteristics and strengths, normalized counts across all probes were not identical and comparisons in expression levels between probes could not be made. It was only appropriate to compare counts from the same probe across different test samples. Each probe for all 3 cell lines detected transcripts, demonstrating that all cell lines retain all exons at the PTEN locus and that the PTEN probes performed as expected. The NCI-H460 cell line had high transcript abundance and the Calu-3 cell line had low transcript abundance which was consistent with their reported levels of protein expression. Even though PTEN protein was not detected in the LNCaP cell line it nonetheless harbored PTEN transcripts. It is known that the PTEN gene in LNCaP carries a frameshift mutation which is responsible for the loss of PTEN protein, but still the gene is transcribed as evident by the results shown in FIG. 3A.

To demonstrate that the PTEN probes can function independently and can detect the presence or absence of individual exons, the probe set was next used to examine 3 cell lines with no PTEN protein expression and known deletions within the PTEN gene; the melanoma cell line HT-144 in which exon 3 is deleted, the melanoma cell line COLO829 in which exon 6 is deleted (ATCC), and the prostate carcinoma cell line PC-3 in which exons 3-9 are deleted. RNA extracted from these 3 cell lines was interrogated by NANOSTRING NCOUNTER with the PTEN code set and the normalized transcript counts for each PTEN probe are shown in FIG. 3B. The results demonstrate that the loss of expression detected by the PTEN exon probes is consistent with the known genomic deletions of these cell lines. Cell line HT-144 showed the highest level of expression across all 9 probes although the PTEN exon 3-4 probe exhibits reduced expression relative to the other probes. It was unexpected for this probe to show no expression, even though exon 3 was deleted from the PTEN gene, because the probe to detect it is a hybrid that extends through exon 4 and into exon 5 due to the small size of exons 3 and 4 and the target sequences fixed at 100 nucleotides. Cell line COLO829 has a deletion for exon 6 and it is clear from the results that expression was detected for all exons except exon 6. Expression for cell line PC-3 was only detected for exon 1 and 2 which was consistent with the PTEN gene being truncated with the loss of exons 3-9. These results were consistent with the specificity of individual PTEN exon probes for their designed exon targets.

In order to measure the PTEN mRNA loss, one must know what the PTEN mRNA expression level in the normal prostate is. Since it is difficult to obtain paired normal and tumor prostate samples from the same patient, the PTEN mRNA expression range in normal prostate was examined by testing each of the nine PTEN probes across multiple independent normal prostate tissues.

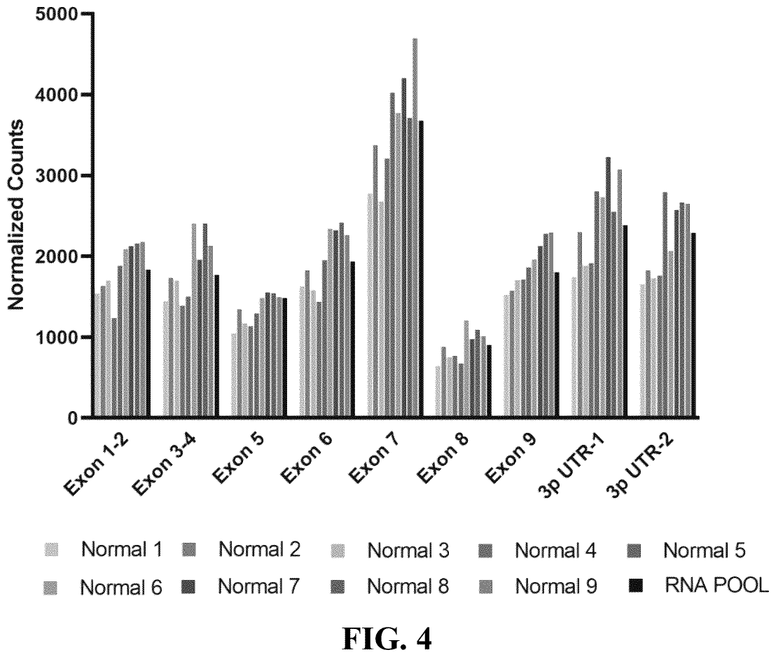

FIG. 4 presents the PTEN expression results obtained using the RNA extracted from 9 normal FFPE tissue samples. Variation appeared between tissues for each of the PTEN probes, but the pattern of expression levels across the 9 probes was consistent across all 9 tissues. For all tissues the expression detected by PTEN exon 7 probe was universally the highest whereas the expression detected by the PTEN exon 8 probe was consistently the lowest. It should be noted that a probe to detect expression of the PTEN pseudogene PTENP1 was also included in the CodeSet containing the 9 PTEN probes and expression was always negligible (data not shown). Since there is high sequence conservation between PTEN and PTENP1, expression detected by the 9 PTEN probes could potentially be due to PTENP1 expression but failure of the PTENP1 specific probe to detect expression can rule out this possibility.

To establish a consistent PTEN mRNA reference as the benchmark to determine the PTEN mRNA loss in the test samples, it was decided to create a NORMAL RNA POOL of 11 RNA samples in equal quantity using RNA extracted from 11 independent patient normal prostate tissues to compensate for the variability of expression between samples. This would serve as a reference baseline of PTEN expression to which the expression levels detected by the PTEN probe set from prostate tumor samples would be compared.

IV.C.2. Converting Normalized Counts from NANOSTRING Data into log₂ Ratio Scores

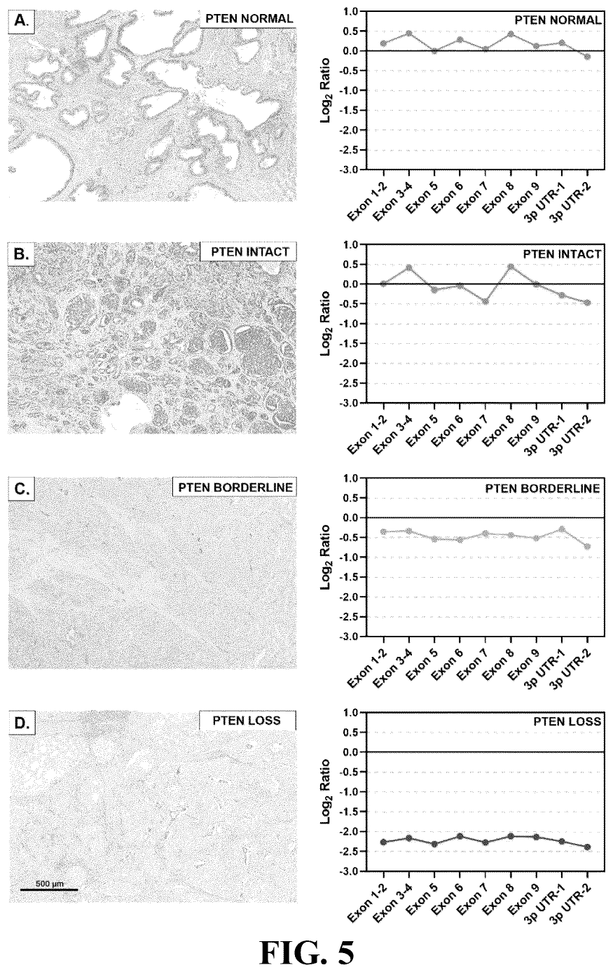

To transform the normalized counts obtained from the NANOSTRING data for the 9 PTEN probes from each prostate tumor tissue into a scale which was more amenable to comparisons between probes, a new measure was devised, which was named “Log₂ Ratio” which equals the Log₂ of the ratio of the normalized count of each individual probe for the test sample to the normalized count of that same probe for the NORMAL RNA POOL. Thus, for each probe: Log₂ Ratio=Log₂ [normalized counts of the test sample/normalized NORMAL RNA POOL counts]. FIG. 5 shows representative examples of the Log₂ Ratio scores across all PTEN probes for a prostate tissue that is normal (FIG. 5, Panel A), a tumor with a PTEN status of INTACT as determined by IHC (FIG. 5, Panel B), a tumor with a PTEN status of BORDERLINE as determined by IHC (FIG. 5, Panel C), and a tumor with a PTEN status of LOSS as determined by IHC (FIG. 5, Panel D). The IHC image of each tissue stained with the PTEN (SP218) antibody is provided adjacent to its respective Log₂ Ratio scores for all 9 PTEN probes. The RNA samples for the three prostate tumors were extracted from whole slices that included adjacent normal stroma and were not from tumor tissue scraped to exclude normal prostate. Converting the normalized count scores for each PTEN probe into the Log₂ Ratio permits direct comparison of the relative values across all probes. Consistent with the IHC status of the tissues, as the IHC status drops from INTACT to LOSS, the Log₂ Ratio scores also drop. This concordance between PTEN mRNA expression measured by NANOSTRING and PTEN protein level determined by IHC suggested that it might be possible to develop an algorithm for assessing the PTEN protein status of prostate tumor tissue from NANOSTRING-derived expression data. This would in turn make it possible to establish a cut-off threshold between PTEN INTACT and PTEN LOSS that provides maximal accuracy with high sensitivity and specificity for predicting PTEN status.

### IV.C.3. Developing an Algorithm and Cut-Off for Predicting PTEN Protein Status of Prostate Tumors

To identify the optimal algorithm and cut-off to facilitate the differentiation of PTEN LOSS from PTEN INTACT from NANOSTRING expression data, ROC (receiver operating characteristic) curve analyses for evaluating classification models (i.e., different probe combinations with potential optimal cut-off) were conducted using PTEN IHC protein status data as the gold standard. The training sample set and the test sample set were used for the algorithm and cut-off development. The training sample set contained 42 cases (scraped-tumor specific) with 23 PTEN INTACT samples, 14 PTEN LOSS samples and 5 BORDERLINE samples (1 BORDERLINE INTACT, 4 BORDERLINE LOSS). The initial algorithm and cut-off development used the NANOSTRING-derived data (Log₂ Ratio of tumor case PTEN probe to RNA NORMAL POOL PTEN probe) generated from the 9 PTEN exon probes (Exon 1-2, 3-4, 5, 6, 7, 8, 9, 3′UTR-1, 3′UTR-2). Each model used the mean value of the Log₂ Ratio scores for the probes included in that model. In addition, exon probe 9 was always included in each model because it was the probe that exhibited the smallest zone of overlap for Log₂ Ratio score between LOSS and INTACT cases assigned by IHC status. To identify the minimal and the most effective probe or probes as part of the algorithm selection, all possible combinations of PTEN exon probes were tested. Five top probe combinations (models) were identified based on sensitivity, specificity and accuracy (Table 2). As set forth in a Table 2, “Sensitivity” equals the percentage of cases accurately identified as LOSS, “Specificity” equals the percentage of cases accurately identified as INTACT, and “Accuracy” equals the overall success in accurately identifying cases as LOSS or INTACT at that Optimal Cut-off. While calculating the Sensitivity, Specificity, and Accuracy, the cases with a status of BORDERLINE INTACT were pooled with INTACT cases; and cases with the status of BORDERLINE LOSS were pooled with LOSS cases. Since Sensitivity and Specificity are of equal diagnostic importance, the optimal cut-off for the probe combination was determined by maximizing the Youden Index.

TABLE 2
Top 5 PTEN probe models from ROC
analysis on training sample set.

| Top 5 | | Optimal | Sensitivity | Specificity | |
|---|---|---|---|---|---|
| Models | Exon Probes | Cutoff | (LOSS) | (INTACT) | Accuracy |
| 1 | 7, 9 | −0.89463 | 72.2% | 91.7% | 0.8333 |
| | | | (13/18) | (22/24) | (35/42) |
| 2 | 1-2, 6, 7, 9 | −0.90254 | 77.8% | 83.3% | 0.8095 |
| | | | (14/18) | (20/24) | (34/42) |
| 3 | 5, 6, 7, 9 | −0.91657 | 77.8% | 83.3% | 0.8095 |
| | | | (14/18) | (20/24) | (34/42) |
| 4 | 7, 9, 3′ UTR-1 | −0.96869 | 72.2% | 87.5% | 0.8095 |
| | | | (13/18) | (21/24) | (34/42) |
| 5 | 5, 7, 9 | −0.90638 | 72.2% | 87.5% | 0.8095 |
| | | | (13/18) | (21/24) | (34/42) |

The five top models and cut-offs were next applied to the test sample set of 21 cases (not scraped-whole section) to evaluate their prediction performance. This dataset included 9 PTEN INTACT samples, 10 PTEN LOSS samples and 2 BORDERLINE samples (1 BORDERLINE INTACT and 1 BORDERLINE LOSS). Model #3 provided the optimal algorithm and cut-off based on the Sensitivity, Specificity and Accuracy by mean signal of the exon probes (Table 2). This model includes the combination of 4 probes (PTEN exon probes 5, 6, 7, 9) in the algorithm and establishes −0.91657 as the cut-off. If the Mean Log₂ Ratio signal for exon probes 5, 6, 7, and 9 is greater than or equal to −0.91657 then the case is predicted to be INTACT, otherwise the case is predicted to be LOSS. Rounding the cut-off to −0.92 does not alter the outcomes (data not shown). Based on these results, Model #3 was selected as the preferred algorithm and cut-off for predicting the PTEN protein status of prostate tumors from NANOSTRING-derived data.

TABLE 3
Prediction performance of top 5 PTEN
probe models on test sample set.

| Top 5 | | Optimal | Sensitivity | Specificity | |
|---|---|---|---|---|---|
| Models | Exon Probes | Cutoff | (LOSS) | (INTACT) | Accuracy |
| 1 | 7, 9 | −0.89463 | 72.7% | 90.0% | 0.810 |
| | | | (8/11) | (9/10) | (17/21) |
| 2 | 1-2, 6, 7, 9 | −0.90254 | 72.7% | 100.0% | 0.857 |
| | | | (8/11) | (10/10) | (18/21) |
| 3 | 5, 6, 7, 9 | −0.91657 | 81.8% | 100.0% | 0.905 |
| | | | (9/11) | (10/10) | (19/21) |
| 4 | 7, 9, 3′ UTR-1 | −0.96869 | 81.8% | 90.0% | 0.857 |
| | | | (9/11) | (9/10) | (18/21) |
| 5 | 5, 7, 9 | −0.90638 | 81.8% | 90.0% | 0.857 |
| | | | (9/11) | (9/10) | (18/21) |

The Model #3 algorithm with a −0.92 cut-off was then used to evaluate an expanded 48-case sample set comprised of the 42-case training set with scraped tumor-specific samples plus an additional 6 scraped tumor-specific samples (Table 3). Although there was a slight decrease in Sensitivity with the addition of 6 cases, both the Specificity and Accuracy of the model showed improvement.

TABLE 4
Prediction performance of Model #3 on a 48-case sample set.

| Top 5 | | Optimal | Sensitivity | Specificity | |
|---|---|---|---|---|---|
| Models | Exon Probes | Cutoff | (LOSS) | (INTACT) | Accuracy |
| 3 | 5, 6, 7, 9 | −0.92 | 77.3% | 84.6% | 0.8125 |
| | | | (17/22) | (22/26) | (39/48) |

IV.C.4. Correlation Analysis of Mean Log₂ Ratio Scores for mRNA Levels and IHC Protein Status

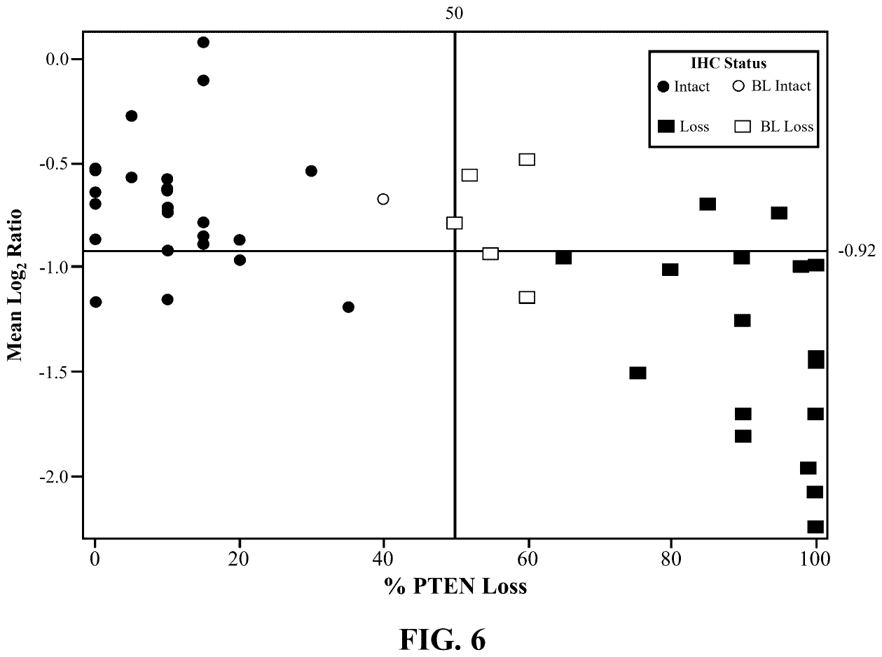

The Pearson Correlation Coefficient for estimating linear dependency between IHC-determined % PTEN loss and the Mean Log₂ Ratio signal for exon probes 5, 6, 7, and 9 was measured in the 48-case sample set. The Correlation Coefficient is −0.66507 (p<0.0001), which shows a strong negative correlation between these two measurements (FIG. 6). This indicates that if a prostate tumor sample has a higher Mean Log₂ Ratio signal, then it will have a lower % PTEN loss.

### IV.C.5. No Significant Difference in Mean Log₂ Ratio Scores Between RNA Isolation Methods

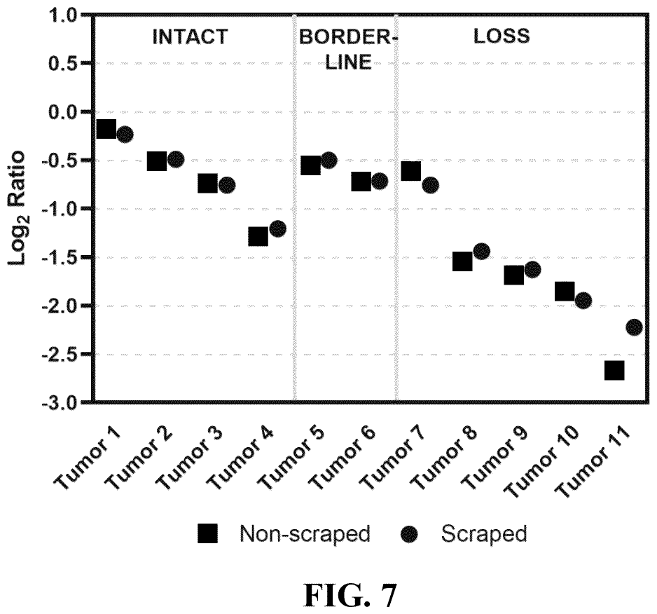

Of the two sample sets that were created with RNA extracted from FFPE prostate adenocarcinoma tissues, one set consisted of 21 cases where the RNA was extracted from whole slices which included both tumor tissue and normal surrounding stroma, whereas the second set of what was initially 42 cases and later extended to 48 cases included only RNA that was extracted from tumor tissue where the surrounding normal stroma had been scraped off the slides to which the sections were attached. It was decided to not combine samples prepared by these 2 distinct methods of collection in the same data set because of the differences in PTEN expression that could be expected with the inclusion of normal prostate tissue surrounding the tumor in the unscraped samples. RNA was extracted from tumor samples that were both scraped and unscraped for 11 prostate tumors, 4 cases with PTEN status of INTACT, 2 cases with PTEN status of BORDERLINE, and 5 cases with PTEN status of LOSS as determined by IHC, to demonstrate whether there was an obvious difference in the PTEN expression levels measured by NanoString. FIG. 7 shows the results for these prostate tumors. As can be seen, the Mean Log₂ Ratio values for each tissue are surprisingly similar and the difference was not significant as determined by a Wilcoxon Signed-Rank Test with a p-value of 0.7002. Therefore, the method of RNA collection appears to not greatly skew the results, demonstrating that loss of PTEN expression in the tumor is sufficient to be detectable by NANOSTRING NCOUNTER despite the presence of PTEN transcripts in the RNA from adjacent normal prostate tissue.

### IV.D. Discussion

The standard methods for detecting PTEN loss in various stages of prostate cancer are IHC and ISH of FFPE tissue. However, the performance of the IHC and ISH assays is often dependent on the quality of patient samples and pre-analytical methods employed prior to the prostate cancer tissues reaching the laboratory. In many instances, assigning PTEN protein status by IHC to borderline cases (samples with staining close to the cut-off threshold of the IHC scoring algorithms) is a challenge. Molecular tests using the same FFPE samples are sometimes more sensitive, exact, rapid and reproducible, depending on the type of technology used. Combining IHC data from anatomic pathology and molecular technologies improves the predictive capability for identification and stratification of PTEN-based prostate cancer. Archival FFPE tissue can be challenging due to the degradation of nucleic acids over time, thus a major advantage of using the NANOSTRING NCOUNTER for gene expression profiling is that robust measures of mRNA abundance are obtained even with low-quality RNA samples. Our algorithm, developed with PTEN mRNA expression derived from NANOSTRING NCOUNTER data, accurately determines a cut-off score to differentiate between PTEN INTACT and LOSS protein status which was previously challenging with IHC data alone.

While the NANOSTRING NCOUNTER gene expression platform detects hundreds of unique mRNA molecules with the use of target-specific probe pairs, most studies utilize a single probe pair per gene. Unfortunately, the reliance on single probe pairs to measure the expression of a specific gene can be risky since the performance of each probe pair may not be optimal and the expression determined by two unique probe pairs for a single gene have often been found to deviate considerably. Therefore, a collection of nine probe pairs designed to identify specific sequences dispersed throughout the entire PTEN gene transcript including all nine exons and 3′ UTR was tested, and this testing confirmed that each unique probe in the probe set demonstrated strong PTEN expression in normal prostate tissue. Using a collection of probes instead of a single random probe thus ensured that detection of the PTEN transcript of interest from FFPE tissue derived mRNA would be robust and provide an accurate measure of PTEN expression in prostate adenocarcinoma tissue.

This study demonstrates that the loss or reduction of PTEN transcripts in prostate adenocarcinomas assessed with the NANOSTRING NCOUNTER is concordant with PTEN protein expression assessed by IHC. Nonetheless, single PTEN probes alone were not good predictors of PTEN protein status. In contrast, the use of multiple probes to detect PTEN mRNA expression levels lent itself to the development of an algorithm that is highly predictive for PTEN protein status and the assignment of a cut-off score to differentiate LOSS from INTACT that provides high sensitivity, specificity and accuracy. By testing multiple models that used the mean of various combinations of the Log₂ Ratio scores for the 9 PTEN probes, an optimal algorithm was selected that takes the mean of the four PTEN exon probes 5, 6, 7 and 9 (=Mean Log₂ Ratio) and sets the cut-off value at −0.92 between PTEN INTACT and PTEN LOSS. There is a strong negative correlation between this Mean Log₂ Ratio score and the percent PTEN protein LOSS as determined by IHC in prostate tumors. This algorithm is sufficiently robust that scraping the tumor area in FFPE tissue for RNA extraction to remove adjacent normal prostate tissue is not essential.

Although the Sensitivity, Specificity and Accuracy of the Mean Log₂ Ratio score is high, there were nonetheless cases that were not accurately predicted by the Mean Log₂ Ratio algorithm and cut-off to have PTEN Intact or Loss status. There are potential explanations for these exceptions. For example, prostate tumors with a low Mean Log₂ Ratio score but a PTEN Intact status could be the consequence of PTEN protein accumulating in the tumor cells with low PTEN transcription due to reduced PTEN protein degradation. In converse, prostate tumors with a high Mean Log₂ Ratio score but a PTEN Loss status can be the result of stop codons introduced in the coding sequence due to nonsense or frameshift mutations resulting in total PTEN protein absence or a truncated protein that is not detected by the antibody used in the IHC assay. Finally, both low Mean Log₂ Ratio but PTEN Intact status, or high Mean Log₂ Ratio but PTEN Loss status in a prostate tumor can be the consequence of mis-regulation of post-transcriptional or post-translational modifications in the cancer cell.

### III.E. Conclusions

In principle, this approach to algorithm and cut-off development employing multiple PTEN probes with NANOSTRING NCOUNTER expression analysis may be extended to other tumor tissues from other organs where PTEN status is a critical prognostic marker in order to provide a predictive measure of PTEN protein abundance. For each new indication a NORMAL RNA POOL specific to that tissue should be assembled to calculate the Log₂ Ratio scores from normalized counts as well as a new algorithm and cutoff can be established. Although the loss of individual exons in PTEN transcripts was not detected in the prostate tumor samples included in this investigation, this PTEN probe set could also be used to identify the loss of individual PTEN exons in other tumor indications. In addition, this study can serve as a model for using the NANOSTRING NCOUNTER and designing multiple probes for detecting transcripts for other specific marker genes encoding proteins that are either over or under expressed in other tumor types where mRNA expression levels can be assessed. From this data an optimal algorithm and cut-off with high predictive value for protein status can be developed. This information can serve as an important new tool to complement and support not only IHC, but additional molecular assays in cancer diagnostics.

### REFERENCES

The following references are incorporated herein by reference in their entirety.
Bhattacharya et al., An approach for normalization and quality control for NanoString RNA expression data, Briefings in Bioinformatics, 2021, Vol. 22, Issue 3: bbaa163.
Brumbaugh et al., NanoStriDE: normalization and differential expression analysis of NanoString nCounter data, BMC Bioinformatics, 2011, Vol. 12, Art. 479.
Brunstein, Molecular methods without nucleic acid amplification, Oct. 1, 2015, Medical Laboratory Observer (available at mlo-online.com).
Cheng et al., Identification of Reference Genes across Physiological States for qRT-PCR through Microarray Meta-Analysis, PLOS ONE, 2011, 6(2): e17347.
Chervoneva et al., Selection of optimal reference genes for normalization in quantitative RT-PCR, BMC Bioinformatics, 2010, Vol. 11, Art. 253.
Dheda et al., Validation of housekeeping genes for normalizing RNA expression in real-time PCR, BioTechniques, 2018, Vol. 37, No. 1.
Eisenberg & Levanon, Human housekeeping genes, revisited, Trends in Genetics, 2013, Vol. 29, Issue 10, pp. 569-574.
Erickson et al., Assessment of normalization strategies for quantitative RT-PCR using microdissected tissue samples, Laboratory Investigations, 2007, Vol. 87, pp. 951-962.
Geiss et al., Direct multiplexed measurement of gene expression with color-coded probe pairs, Nature Biotechnology, 2008, Vol. 26, Issue 3, pp. 317-325.
Jia et al., RCRnorm: An integrated system of random-coefficient hierarchical regression models for normalizing NanoString nCounter data, Annals of Applied Statistics, 2019, Vol. 13, pp. 1617-47.
Jo et al., Conventionally used reference genes are not outstanding for normalization of gene expression in human cancer research, BMC Bioinformatics, 2019, Vol. 20, Supplement 10, Article 245.
Krasnov et al., Differentially Expressed Genes Associated With Prognosis in Locally Advanced Lymph Node-Negative Prostate Cancer, Frontiers in Genetics, 2019, Vol. 10.
Li et al., Comparing the normalization methods for the differential analysis of Illumina high-throughput RNA-Seq data, BMC Bioinformatics, 2015, Vol. 16, Art. 347.
Molania, R., et al., 2019, Nucleic Acids Research 47, 6073-6083. NanoString Technologies, Inc., Technical Note: Reference Genes for Normalization of Expression Data, available at nanostring.com (last accessed 5 Apr. 2023) (“NanoString Technical Note”).
Sharan et al., Consensus reference gene(s) for gene expression studies in human cancers: end of the tunnel visible?, Cellular Oncology, 2015, Vol. 38, Issue 6, pp. 419-431.
Silvia et al., Selection of suitable reference genes for accurate normalization of gene expression profile studies in non-small cell lung cancer, BMC Cancer, 2006, Vol. 6, Art. 200.
Waggott et al., NanoStringNorm: an extensible R package for the pre-processing of NanoString mRNA and miRNA data, Bioinformatics, 2012, Vol. 28, Issue 11, pp. 1546-1548.
Wang et al., NanoStringDiff: a novel statistical method for differential expression analysis based on NanoString nCounter data, Nucleic Acids Research, 2016, Vol. 44, Issue 20, Art. e151.
Zhu et al., On the nature of human housekeeping genes, Trends in Genetics, 2008, Vol. 24, Issue 10, pp. 481-484.
Zollinger et al., GeoMx™ RNA Assay: High Multiplex, Digital, Spatial Analysis of RNA in FFPE Tissue, Methods in Molecular Biology, 2020, Issue 2148, pp. 331-45.

1. A method of determining an expression status of a protein in a test sample and/or a diagnostic status of the test sample, the method comprising:
obtaining a normalized quantity for each of at least two distinct regions of a messenger ribonucleic acid (mRNA) transcript encoding the protein of interest in a test sample (N_{Test});
obtaining either:
a normalized reference quantity for each of the at least two distinct regions of the mRNA transcript in a reference sample (N_{Ref}); or
a non-normalized quantity of each of the at least two distinct regions of the mRNA transcript in a reference sample (Q_{Ref}), wherein the reference sample comprises a known quantity of the mRNA; or
for each distinct region of the at least two distinct regions of the mRNA, generating a ratio between the N_{Test} and either N_{Ref} or Q_{Ref} to obtain a set of region ratios; and
computing with a trained classifier a score indicative of the expression status of the protein in the test sample and/or the diagnostic status of the test sample using the obtained set of region ratios.

2. The method of claim 1, wherein the reference sample is derived from a plurality of samples each having a known expression status for the protein and/or a known diagnostic status and wherein:
the N_{Test} is obtained for each region of the mRNA by:
obtaining a non-normalized quantity of each region of the mRNA transcript in the test sample (Q_{Test});
measuring a set of housekeeping mRNA in the test sample to obtain a test normalization factor (HK_{Test}); and
normalizing Q_{Test} against HK_{Test} to obtain N_{Test}; and
wherein N_{Ref} is obtained for each region of the mRNA by:
obtaining a non-normalized quantity of each region of the mRNA in the reference sample (Q_{Ref});
measuring the set of housekeeping mRNA in the reference sample to obtain a reference normalization factor (HK_{Ref}); and
normalizing Q_{Ref} against HK_{Ref} to obtain N_{Ref}.

3. The method of claim 2, wherein the reference sample has a known quantity of the mRNA, and the set of region ratios comprises the ratios between N_{Test} and Q_{Ref}.

4. The method of claim 1, further comprising comparing the computed score to one or more pre-determined cutoffs to assign the expression status to of protein.

5. The method of claim 1, wherein the mRNA transcript is selected from the group consisting of a human PTEN mRNA, a human MYC mRNA, a human TROP2 mRNA, a human CRBN mRNA, a human AR mRNA, a human AXL mRNA, a human DLL3 mRNA, a human FGFR2IIIb mRNA, a human FOLR1 mRNA, a human HER2 mRNA, a human LAG3 mRNA, a human MET mRNA, a human TIGIT mRNA, and a human TP53 mRNA.

6. The method of claim 5, wherein the mRNA is a PTEN mRNA and the regions of the PTEN mRNA are selected from the group consisting of an exon 1 region, an exon 2 region, an exon 1-2 region, an exon 3 region, an exon 4 region, an exon 3-4 region, an exon 5 region, an exon 6 region, an exon 7 region, an exon 8 region, an exon 9 region, and a 3′ UTR region.

7. The method of claim 6, wherein the exon 1-2 region comprises SEQ ID NO: 1, the exon 3-4 region comprises SEQ ID NO: 2, the exon 5 region comprises SEQ ID NO: 3, the exon 6 region comprises SEQ ID NO: 4, the exon 7 region comprises SEQ ID NO: 5, the exon 8 region comprises SEQ ID NO: 6, the exon 9 region comprises SEQ ID NO: 7, 3′ UTR region comprises SEQ ID NO: 8 or SEQ ID NO: 9.

8. The method of claim 6, wherein the regions of the PTEN mRNA comprises:
(a) the exon 7 region and the exon 9 region;
(b) the exon 1-2 region, the exon 6 region, the exon 7 region, and the exon 9 region;
(c) the exon 5 region, the exon 6 region, the exon 7 region, and the exon 9 region;
(d) the exon 7 region, the exon 9 region, and 3′ UTR region; or
(e) the exon 5 region, the exon 7 region, and the exon 9 region.

9. The method of claim 1, wherein the set of region ratios comprises a log ratio.

10. The method of claim 1, wherein the set of region ratios comprises a log₂ ratio.

11. A method comprising:
(a) performing a quantitative or semi-quantitative nucleic acid detection method on a test sample comprising mRNA or cDNA, wherein the quantitative or semi-quantitative nucleic acid detection method comprises:
(a1) contacting the test sample with a probe set comprising:
(a1a) a plurality of distinctly labeled probes or probe pairs specific for distinct regions of the same mRNA or corresponding cDNA; and
(a1b) one or more distinctly labeled probes or probe pairs specific for a housekeeping mRNA or corresponding cDNA; and
(a2) detecting and quantifying each distinctly labeled probe or probe pair that specifically hybridizes to the distinct regions of the same mRNA or the corresponding cDNA to obtain a quantity Q_{Test}, and
(a3) detecting and quantifying each distinctly labeled probe or probe pair that specifically hybridizes to the housekeeping mRNA to obtain a quantity HK_{Test}; and
(b) performing a quantitative or semi-quantitative nucleic acid detection method on a reference sample comprising mRNA or cDNA, wherein the quantitative or semi-quantitative nucleic acid detection method comprises:
(b1) contacting the reference sample with the probe set;
(b2) detecting and quantifying each probe or probe pair that specifically hybridizes to the distinct regions of the same mRNA or the corresponding cDNA to obtain a quantity Q_{Ref}, and
(b3) detecting and quantifying each probe or probe pair that specifically hybridizes to the housekeeping mRNA to obtain a quantity HK_{Ref},
wherein the distinct regions of the same mRNA are different exons or groups of exons, 5′ untranslated regions, and/or 3′ untranslated regions.

12. The method of claim 11, wherein the probe set comprises a distinctly labeled probe or probe pair specific for at least 2 exons of the mRNA or corresponding cDNA.

13. The method of claim 11, wherein the probe set comprises a distinctly labeled probe or probe pair specific for greater than 2 exons of the mRNA or corresponding cDNA, but less than the entire set of exons contained in the mRNA or the corresponding cDNA.

14. The method of claim 11, wherein the test sample is derived from a tumor sample, and the distinct regions of the same mRNA or corresponding cDNA are PTEN mRNA or PTEN cDNA.

15. The method of claim 14, wherein the distinct regions of the PTEN mRNA or PTEN cDNA are selected from the group consisting of an exon 1 region, an exon 2 region, an exon 1-2 region, an exon 3 region, an exon 4 region, an exon 3-4 region, an exon 5 region, an exon 6 region, an exon 7 region, an exon 8 region, an exon 9 region, and a 3′-UTR region.

16. The method of claim 14, wherein the distinct regions of the PTEN mRNA or PTEN cDNA are selected from the group consisting of any one of SEQ ID NOS: 1-9 and complements thereof.

17. The method of claim 14, wherein the distinct regions of the PTEN mRNA or PTEN cDNA comprises:
an exon 7 region and an exon 9 region;
an exon 1-2 region, an exon 6 region, an exon 7 region, and an exon 9 region;
an exon 5 region, an exon 6 region, an exon 7 region, and an exon 9 region;
an exon 7 region, an exon 9 region, and 3′ UTR region; or
an exon 5 region, an exon 7 region, and an exon 9 region.

18. The method of claim 14, wherein the distinct regions of the PTEN mRNA or PTEN cDNA comprises:
SEQ ID NO: 5 and SEQ ID NO: 7;
SEQ ID NO: 1, SEQ ID NO: 4, SEQ ID NO: 5, and SEQ ID NO: 7;
SEQ ID NO: 3, SEQ ID NO: 4, SEQ ID NO: 5, and SEQ ID NO: 7;
SEQ ID NO: 5, SEQ ID NO: 7, and SEQ ID NO: 8; or
SEQ ID NO: 3, SEQ ID NO: 5, and SEQ ID NO: 7.

19. The method of claim 11, further comprising:
(d) for each distinct region of the mRNA, normalizing each Q_{Test} against HK_{Test} to obtain a normalized quantity of each probe or probe pair that specifically hybridizes to the distinct regions of the same mRNA or the corresponding cDNA in the test sample (N_{Test});
(e) for each distinct region of the mRNA, normalizing each Q_{Ref} against HK_{Ref} to obtain a normalized quantity of each probe or probe pair that specifically hybridizes to the distinct regions of the same mRNA or the corresponding cDNA in the reference sample (N_{Ref});
(f) generating a region ratio between each N_{Test} and N_{Ref} for each distinct region in the same mRNA or the corresponding cDNA to obtain a set of region ratios; and
(g) computing with a trained classifier a composite score indicative of a protein expression status of the test sample or a diagnostic status of the test sample, wherein the computing of the composite score is based on the generated region ratio between each N_{Test} and N_{Ref}.

20. A kit for quantifying a target messenger RNA (mRNA) molecule or a target complementary DNA (cDNA) molecule, the kit comprising (i) a plurality of nucleic acid probes complementary to distinct, non-overlapping target regions of the same target mRNA molecule or target cDNA molecule; and (ii) a set of housekeeping probes, wherein each housekeeping probe is complementary to a target region of a housekeeping mRNA molecule or cDNA molecule, wherein the target regions are exons, groups of adjacent exons, and/or untranslated regions.


SEQUENCE LISTING (9 sequences)

SEQ ID NO: 1 - RNA, 100 nt, Homo sapiens
  source 1..100: mol_type = mRNA
aggagatatcaagaggatggattcgacttagacttgacctatatttatccaaacattattgctatgggatttcctgcagaaagacttgaaggcgtataca

SEQ ID NO: 2 - RNA, 100 nt, Homo sapiens
  source 1..100: mol_type = mRNA
tggattcaaagcataaaaaccattacaagatatacaatctttgtgctgaaagacattatgacaccgccaaatttaattgcagagttgcacaatatccttt

SEQ ID NO: 3 - RNA, 100 nt, Homo sapiens
  source 1..100: mol_type = mRNA
catgttgcagcaattcactgtaaagctggaaagggacgaactggtgtaatgatatgtgcatatttattacatcggggcaaatttttaaaggcacaagagg

SEQ ID NO: 4 - RNA, 100 nt, Homo sapiens
  source 1..100: mol_type = mRNA
agtaactattcccagtcagaggcgctatgtgtattattatagctacctgttaaagaatcatctggattatagaccagtggcactgttgtttcacaagatg

SEQ ID NO: 5 - RNA, 100 nt, Homo sapiens
  source 1..100: mol_type = mRNA
gatatattcctccaattcaggacccacacgacgggaagacaagttcatgtactttgagttccctcagccgttacctgtgtgtggtgatatcaaagtagag

SEQ ID NO: 6 - RNA, 100 nt, Homo sapiens
  source 1..100: mol_type = mRNA
atcgatagcatttgcagtatagagcgtgcagataatgacaaggaatatctagtacttactttaacaaaaaatgatcttgacaaagcaaataaagacaaag

SEQ ID NO: 7 - RNA, 100 nt, Homo sapiens
  source 1..100: mol_type = mRNA
atgttagtgacaatgaacctgatcattatagatattctgacaccactgactctgatccagagaatgaaccttttgatgaagatcagcatacacaaattac

SEQ ID NO: 8 - RNA, 100 nt, Homo sapiens
  source 1..100: mol_type = mRNA
actagctgtggtctgacctagttaatttacaaatacagattgaataggacctactagagcagcatttatagagtttgatggcaaatagattaggcagaac

SEQ ID NO: 9 - RNA, 100 nt, Homo sapiens
  source 1..100: mol_type = mRNA
ttggatgtgcagcagcttacatgtctgaagttacttgaaggcatcacttttaagaaagcttacagttgggccctgtaccatcccaagtcctttgtagctc